SEQUENCE LISTING (42 sequences)

SEQ ID NO: 1 - DNA, 1732 bp, Punica granatum
cggcggactctgtcagttgcatgagccatttccgcacgtcacccctctctctctctctgctcctctcttcagctcccacgccggaaccttttcgtcaacaatccggatggcgacctccgacggcaacgagagcttaggcgccgcggattccgcttccagcctcagcgccgactcagatccgcccacctcctccctcaggcgacggcacgcccccgcgcctgcagctgcagctgctgtgtctgcgttcgagtcggagaatctggtcaaggatttgtgcgatcgcagctgcgaggcttcaccgtcacctagctccgacgacggcaacgatcctcgcagcctcggtgctgaccatggctctgtggattccgcgaattctgttagcgatggcggtagtgttaaggatgagaagattggtgtttccggtgagaaatcgggattggagcatgccgtcaagttcgcctaccgtgcatcgtctccagctcaccggaaagtgaaggagagccctcttagttctagtaatatcttcaagcagagtcacgcgggtctcttcaacctctgtatagttgttcttgttgctgtaaacagcagacttatcatcgagaatctaatgaagtatggttggttaataaagactggtttttggttcagttcgagatcattaagagactggccactcttcatgtgttgccttagtcttcccattttcccccttgctgctttcctggttgagaagttggtgcagcgaaattgtctgtcagaaccagttgttgtttgttctcatatattcattacttctgcgtccattctttatcctgtttttgtgattctgaggtgtgattctgctgttcaatctggtgttgctctaatgctcttcgcttgcattgtgtggcttaaattggtgtcatatgcgcacacgaattacgatatgagatcagttgccaagtctcttgataagggcgatgccattgctgatccgataattgtagagcatccttaccgtgtggacttcaaggatttgatatacttcatggttgcacccaccctatgttaccagctttcctatcctcgcacactatgtgtacgcaaaggttgggcagttcgccaagtcattaagttaatattgttcacaggagttatgggctttatcgttgaacaatatatcaatccaatcgtgcaaaattctcaacaccccctgaaagggaatttcttatatgccattgagagagttttgaagctttctgttccaaatttatacgtttggctctgcatgttctactgcttctttcatctctggttaaacatactggcagagcttctctgttttggtgatcgtgaattctataaagactggtggaatgcaaagacagttgaggagtattggaggatgtggaacatgcctgttcataagtggatggtgcggcatatctactttccttgcttgaggaatggaataccaaggggagttgcggttcttattgcctttatagtgtctgcagtgtttcatgagctgtgtattgctgttccatgtcacgtgttcaagccatgggcatttataggaattatgtttcaggttcccttggttttgatcacgacctacctacaaaagaagttccagagctccatggtggggaatatgatcttctggttcatattttgcatcttcggccaacccatgtgtgtgcttctgtactaccatgacttgatgaatcgaaaaggctcccggccgtaatgg

SEQ ID NO: 2 - protein, 540 aa, Punica granatum
MATSDGNESLGAADSASSLSADSDPPTSSLRRRHAPAPAAAAAVSAFESENLVKDLCDRSCEASPSPSSDDGNDPRSLGADHGSVDSANSVSDGGSVKDEKIGVSGEKSGLEHAVKFAYRASSPAHRKVKESPLSSSNIFKQSHAGLFNLCIVVLVAVNSRLIIENLMKYGWLIKTGFWFSSRSLRDWPLFMCCLSLPIFPLAAFLVEKLVQRNCLSEPVVVCSHIFITSASILYPVFVILRCDSAVQSGVALMLFACIVWLKLVSYAHTNYDMRSVAKSLDKGDAIADPIIVEHPYRVDFKDLIYFMVAPTLCYQLSYPRTLCVRKGWAVRQVIKLILFTGVMGFIVEQYINPIVQNSQHPLKGNFLYAIERVLKLSVPNLYVWLCMFYCFFHLWLNILAELLCFGDREFYKDWWNAKTVEEYWRMWNMPVHKWMVRHIYFPCLRNGIPRGVAVLIAFIVSAVFHELCIAVPCHVFKPWAFIGIMFQVPLVLITTYLQKKFQSSMVGNMIFWFIFCIFGQPMCVLLYYHDLMNRKGSRP

SEQ ID NO: 3 - DNA, 1479 bp, Punica granatum
acgaaaccacgaaaatcatcgaagacgagagagatatggcgtcgtgtctggaagcccccagagagagacaacgaaaagctctgatgggcactgtcgttcatttctagtccatcatcgtcaaggacaaaaaactgcttcctctgtttccccggagcagcggattgtgatattgattggtagcagttccgaggttgtgggtcgacagtcttcggtgtgaatctttcgtctttccgccattggagatgggagaggaggcgagtgtgaggctcggtgaggatcagcagaggggagagatggggttgagagtgtttgatgggaggaaggagttcccttcttcgaatacgttttccactctgctcgctctggcgatatggctcggcgctatccactttgatgccgttttggtcctcttctctctcctcttcctccctctctccaaagccctccttgtgttcgggttgcatgtggcttttatggtgatcccaattgatgatcagagcaaggttggtcggagattgtcgaggtacatatgtaggcatgcctgccgctattttccagtgaccctgcatgtggaggactttggatgtttcgttcctaatcgcgcatatgtctttggttatgagccacactcagtgttgcctattggagtcgtggcacttgccgatcttactggttttatgcctctcccgaagatgaaagtcctcgccagcagtgctgtctttatcacgccatttctaagacacatatggacgtggttggggcttacacctgcaactaggaaaaacttcgtctcactcttggagtcaggttatagctgcattgtggtgcccggtggcgtgcaggagacattctacatggagcacggttctgagattgcttttctcaagaagaggagagggttcgtgcgtacagcgatggagaccgggtgctctcttgttccagtcttctgctacggtcaatctcaggtctacgagtggtggaagcctagcggggagttgttcttgaagatctcccgattgatcaagtttacccccatattcttctggggcatgcttgggtctccattgcctttccaacatccgatgcatgtcgtggttgggaaaccgattgaggtgaagagaacttcaaaccctacagcagaagaggttgacaagttgcataaacagtttgttgagtctctacaagagctatttgagcgccacaaggcacgagtcggtcacaaggatctggaactgaagatcctctgattgaagctcttcttgatatcatgtgtatcgaaacagatgtctatttgattagcagcatatcctacatctttcgcttttcgtgttcaattaaatttatctctgtcgataaaatgcggtgtatttgcagaccaaatctcatgcgtgtacctatatttacatgatcaagttattctcaatacaggaccaagtagcagtaagtcacaaaaaaaaaaaaaaaaaaaaaaaaaaaaaa

SEQ ID NO: 4 - protein, 334 aa, Punica granatum
MGEEASVRLGEDQQRGEMGLRVFDGRKEFPSSNTFSTLLALAIWLGAIHFDAVLVLFSLLFLPLSKALLVFGLHVAFMVIPIDDQSKVGRRLSRYICRHACRYFPVTLHVEDFGCFVPNRAYVFGYEPHSVLPIGVVALADLTGFMPLPKMKVLASSAVFITPFLRHIWTWLGLTPATRKNFVSLLESGYSCIVVPGGVQETFYMEHGSEIAFLKKRRGFVRTAMETGCSLVPVFCYGQSQVYEWWKPSGELFLKISRLIKFTPIFFWGMLGSPLPFQHPMHVVVGKPIEVKRTSNPTAEEVDKLHKQFVESLQELFERHKARVGHKDLELKIL

SEQ ID NO: 5 - DNA, 2743 bp, Punica granatum
aatcagagcttaagctctaaggtcctccattaatgacttctccgagctcctccccagcctcccctccctcctcctcctccttacagatcatcactcggtaaaagggtcacccccactagggccccacgatggcgtttctctggcggaggcgggccacctacgccgatgccccccagccaccgtcaccgtcgaaagaatcccactccaagagggacagacacaagaaaggcagcaaaaacgatggcaataaacagaccgaacagccggcccctccgctgcagccgccgcctccgcctccgccgccgccgacgaagaagtggtcgtgcatcgacgggtgctgctggttcatcgggtgcatatgctcgacctggtggctgctgctgttcctctacaatgcgatgccggcgtcgttccctcagtacgtgactgaggcgattacggggccactgcctgacccgcctggggtgaagctccggaaggaggggctgacagcgaagcacccagttgtcttcgtgcccgggatcgtcaccggcgggctcgagttctgggagggccaccattgcgccgatgggctgttcaggaagcgcctctggggtggcaccttcggggagctctataaacgacctttgtgctgggttgagcacatgtcacttgataatgagacgggattggatccttctggaataagggtcaggcctgtttctggtcttgttgcagcggattattttgctccgggttattttgtctgggcggtcttgattgcaaatttggccagactaggatatgaggagaagactatgtacatggctgcatatgactggagactgtcctttcagaacaatgaggtcagagaccagacactcagcaggataaagagcaacatagaggtcatggtggctacaaatggtggtagaaaggtggttgtaataccacactcgatgggtgttctttactttctacatttcatgaagtgggttgaagctccaactcctatgggcggaggaggtggacccgactggtgcgcaaaacacataaaggcagttatgaatattggtgggccatttttaggtgttccaaaagcagttgcaggactcttttctattgaagcaaaagatatagctgttgcccggtcgtttgccccaggtgtattggacaaggacgtctttgggcttcagactctacagcatatgatgaggatgacccgaacttgggattcaaccatgtcaatgataccgaaaggcggtgacactatttggggtggccttgattggtcacctgaaggattcgccagctgtagctcaaaaaggtcaaagaataactatggtaacagcactaaaggtcaccaacgtaagaaggtgaattacgggaggctaatatcgttcgggaaagatatagctgaaacccattcctcaatgattaagaggacagatttcaggggtgaattcaagggccataacctcttaaatacgaccaactgtgatgtttggacagagtaccatgatatgggagtacagggtgtaaaagcaatctcggactacaagatgtacactgcaggttcaatattggaccttctgcactttgttgccccaaagatgatgaagcgtgggagcgcccatttttcatatggaattgccgataatttagacgacccaaagtatcaacactaccgatattggtcgaatccattggaaaccaagttaccaaatgctccggatatggagatatattctatgtacggagtcgggatacccacagaacgagcgtatgtatacaagctaacccccccttccgaatgctacattccttttcagatagacagctctgcggatggttcaggtgacgattcttgtctacaaggtggagttttctcaatcaatggggacgagactgttcccgtcttgagtgcaggctttatgtgtgcgaaagggtggagggggaaaactcgattcaacccctcggggataaggactttgataagggagtatgaccatgccccacccgctaacctgttggagggccggggaacccagagtggggcacatgtggacatcttgggcaactttgcattgattgaggatatcttaagggtggctgccggggccacgggagatgacttggcgggagatcgggtctactcagatatcttcaagtggtctgagaggattgacttgccactctagaagatctattacgtgacaatgtagtccatgtctccattcttgggcaaaacgacaatccgaaccggctctcatagttccccatagccaatagttgggaagcagcaagtccaaacctatacagccgaccagtcattccagaaaaatgaaagggtagggttgaggattttctcgtggcattcgctgaaactaaacagcttcactgtcttggaaaacgctcaaacagctaggaggaagaatcgatatctcactgttgcgcgacggtaggcttcccgtctcctctgtgggttatatcgacttctgtaagatacacgtggtttcccaccttcccgggattctgggcgattggtcaatcaatttatttgattaatttgtccatgaaattttgtattagaactgtaaattctgttataaccgcgggttctataaatttttcgtcctgttttatcaattctccagaactgggtaagcaagcaattgatatccctgttgtgctgcactcctacagatacaaaacaattgaatagcagtggaatttccattacaaaaaaaaaaaaaaaaaaaaa

SEQ ID NO: 6 - protein, 683 aa, Punica granatum
MAFLWRRRATYADAPQPPSPSKESHSKRDRHKKGSKNDGNKQTEQPAPPLQPPPPPPPPPTKKWSCIDGCCWFIGCICSTWWLLLFLYNAMPASFPQYVTEAITGPLPDPPGVKLRKEGLTAKHPVVFVPGIVTGGLEFWEGHHCADGLFRKRLWGGTFGELYKRPLCWVEHMSLDNETGLDPSGIRVRPVSGLVAADYFAPGYFVWAVLIANLARLGYEEKTMYMAAYDWRLSFQNNEVRDQTLSRIKSNIEVMVATNGGRKVVVIPHSMGVLYFLHFMKWVEAPTPMGGGGGPDWCAKHIKAVMNIGGPFLGVPKAVAGLFSIEAKDIAVARSFAPGVLDKDVFGLQTLQHMMRMTRTWDSTMSMIPKGGDTIWGGLDWSPEGFASCSSKRSKNNYGNSTKGHQRKKVNYGRLISFGKDIAETHSSMIKRTDFRGEFKGHNLLNTTNCDVWTEYHDMGVQGVKAISDYKMYTAGSILDLLHFVAPKMMKRGSAHFSYGIADNLDDPKYQHYRYWSNPLETKLPNAPDMEIYSMYGVGIPTERAYVYKLTPPSECYIPFQIDSSADGSGDDSCLQGGVFSINGDETVPVLSAGFMCAKGWRGKTRFNPSGIRTLIREYDHAPPANLLEGRGTQSGAHVDILGNFALIEDILRVAAGATGDDLAGDRVYSDIFKWSERIDLPL

SEQ ID NO: 7 - DNA, 23 bp, Artificial Sequence
  forward F1 primer
gaaatgggagctgatggaacaat

SEQ ID NO: 8 - DNA, 20 bp, Artificial Sequence
  reverse R1 primer
tcagaacttgctcttgaacc

SEQ ID NO: 9 - DNA, 29 bp, Artificial Sequence
  napin promoter, forward F2 primer including EcoRI site
atagaattcaagctttcttcatcggtgat

SEQ ID NO: 10 - DNA, 29 bp, Artificial Sequence
  napin promoter, reverse R2 primer including SmaI site
atacccggggtccgtgtatgtttttaatc

SEQ ID NO: 11 - DNA, 27 bp, Artificial Sequence
  PgFADX, forward F3 primer including SmaI
tatcccgggatgggagctgatggaaca

SEQ ID NO: 12 - DNA, 30 bp, Artificial Sequence
  PgFADX, reverse R3 primer including NotI site
cgcgcggccgctcagaacttgctcttgaac

SEQ ID NO: 13 - DNA, 33 bp, Artificial Sequence
  Nos terminator, forward primer F4 including NotI site
cgccggcggccgcgatcgttcaaacatttggca

SEQ ID NO: 14 - DNA, 30 bp, Artificial Sequence
  Nos terminator, reverse primer R4 including KpnI site
tatggtacccgatctagtaacatagatgac

SEQ ID NO: 15 - DNA, 29 bp, Artificial Sequence
  napin promoter, forward F5 primer including KpnI site
ataggtaccaagctttcttcatcggtgat

SEQ ID NO: 16 - DNA, 30 bp, Artificial Sequence
  napin promoter, reverse R5 primer including XhoI site
atactcgaggtccgtgtatgtttttaatct

SEQ ID NO: 17 - DNA, 26 bp, Artificial Sequence
  PgFAD2, forward F6 primer including XhoI
taactcgagatgggagccggtggaag

SEQ ID NO: 18 - DNA, 28 bp, Artificial Sequence
  PgFAD2, reverse R6 primer including XbaI site
tattctagatcagaggttcttcttgtac

SEQ ID NO: 19 - DNA, 30 bp, Artificial Sequence
  Nos terminator, forward F7 primer including XbaI site
tattctagagatcgttcaaacatttggcaa

SEQ ID NO: 20 - DNA, 30 bp, Artificial Sequence
  Nos terminator, reverse R7 primer including SalI site
atagtcgaccgatctagtaacatagatgac

SEQ ID NO: 21 - DNA, 31 bp, Artificial Sequence
  F14 primer including AscI site
ataggcgcgccaagctttcttcatcggtgat

SEQ ID NO: 22 - DNA, 30 bp, Artificial Sequence; identical to SEQ ID NO: 14
  R14 primer including KpnI

SEQ ID NO: 23 - DNA, 29 bp, Artificial Sequence
  F15 primer including KpnI
ataggtaccaagctttcttcatcggtgat

SEQ ID NO: 24 - DNA, 32 bp, Artificial Sequence
  R15 primer including AscI
tatggcgcgcccgatctagtaacatagatgac

SEQ ID NO: 25 - DNA, 29 bp, Artificial Sequence
  napin promoter, forward F16 primer including AgeI site
ataaccggtaagctttcttcatcggtgat

SEQ ID NO: 26 - DNA, 30 bp, Artificial Sequence; identical to SEQ ID NO: 16
  napin promoter, reverse R16 primer including XhoI site

SEQ ID NO: 27 - DNA, 26 bp, Artificial Sequence
  PgDGAT2, forward F17 primer including XhoI
taactcgagatgggagaggaggcgag

SEQ ID NO: 28 - DNA, 29 bp, Artificial Sequence
  PgDGAT2, reverse R17 primer including XbaI site
atattctagatcagaggatcttcagttcc

SEQ ID NO: 29 - DNA, 30 bp, Artificial Sequence; identical to SEQ ID NO: 19
  Nos terminator, forward F18 primer including XbaI site

SEQ ID NO: 30 - DNA, 32 bp, Artificial Sequence
  Nos terminator, reverse R18 primer including NotI site
cgcgcggccgccgatctagtaacatagatgac

SEQ ID NO: 31 - DNA, 18 bp, Artificial Sequence
  forward F8 primer
atggcgacctccgacggc

SEQ ID NO: 32 - DNA, 19 bp, Artificial Sequence
  reverse R8 primer
ttacggccgggagcctttt

SEQ ID NO: 33 - DNA, 18 bp, Artificial Sequence
  forward F9 primer
atgggagaggaggcgagc

SEQ ID NO: 34 - DNA, 19 bp, Artificial Sequence
  reverse R9 primer
tcagaggatcttcagttcc

SEQ ID NO: 35 - DNA, 19 bp, Artificial Sequence
  forward F10 primer
atggcgtttctctggcgga

SEQ ID NO: 36 - DNA, 20 bp, Artificial Sequence
  reverse R10 primer
ctagagtggcaagtcaatcc

SEQ ID NO: 37 - DNA, 31 bp, Artificial Sequence
  forward F11 primer including BamHI site
aataggatccgaaatgggagctgatggaaca

SEQ ID NO: 38 - DNA, 29 bp, Artificial Sequence
  reverse R11 primer including KpnI site
ttatggtacctcagaacttgctcttgaac

SEQ ID NO: 39 - DNA, 34 bp, Artificial Sequence
  forward F12 primer including NotI site
gcagagcggccgcgaaatggcgacctccgacggc

SEQ ID NO: 40 - DNA, 31 bp, Artificial Sequence
  reverse R12 primer including PacI site
atatttaattaattacggccgggagcctttt

SEQ ID NO: 41 - DNA, 33 bp, Artificial Sequence
  forward F13 primer including NotI site
gcagagcggccgcgaaatgggagaggaggcgag

SEQ ID NO: 42 - DNA, 31 bp, Artificial Sequence
  reverse R13 primer including PacI site
atatttaattaatcagaggatcttcagttcc

(19) United States
(12) Patent Application Publication
(10) Pub. No.: US 2014/0289908 A1
(43) Pub. Date: Sep. 25, 2014

(54) GENE COMBINATIONS FOR PRODUCING PUNICIC ACID IN TRANSGENIC PLANTS

(71) Applicant: The Governors of The University of Alberta, Edmonton (CA)

(72) Inventors: Randall J. WESELAKE, Edmonton (CA); Elzbieta MIETKIEWSKA, Edmonton (CA)

(73) Assignee: The Governors of The University of Alberta, Edmonton (CA)

(21) Appl. No.: 14/224,582

(22) Filed: Mar. 25, 2014

Related U.S. Application Data
(60) Provisional application No. 61/804,877, filed on Mar. 25, 2013.

(51) Int. Cl. C12N 15/82; C12N 9/10

(52) U.S. Cl. CPC C12N 15/8247; C12N 9/1029
USPC 800/306; 536/23.2; 435/320.1; 435/419; 435/415; 435/416; 800/298; 435/412; 800/312; 800/322; 800/320.3; 800/320; 800/320.1

(57) ABSTRACT

Transgenic plants with enhanced punicic acid accumulation result from over-expression of PgFADX and PgFAD2, and optionally, one or more of PgDGAT1, PgDGAT2, and PgPDAT1. The invention also relates to novel isolated Punica granatum diacylglycerol acyltransferases: type 1 (PgDGAT1), type 2 (PgDGAT2) and phospholipid:diacylglycerol acyltransferases (PgPDAT1); polynucleotide sequences encoding the DGATs and PDAT enzymes; polynucleotide constructs, vectors and host cells incorporating the polynucleotide sequences; and methods of producing and using same.

### CROSS-REFERENCE TO RELATED APPLICATIONS

This application claims priority benefit of U.S. Provisional Patent Application No. 61/804,877, filed Mar. 25, 2013, the entire contents of which is incorporated herein by reference.

### FIELD OF THE INVENTION

The present invention relates to transgenic plants with enhanced punicic acid accumulation resulting from over-expression of PgFADX and PgFAD2, and methods of increasing punicic acid production in oilseed plants. The invention further relates to isolated desaturases (FAD2) and fatty acid conjugases (FADX), diacylglycerol acyltransferases: type 1 (DGAT1) and type 2 (DGAT2), and phospholipid:diacylglycerol acyltransferases (PDAT1) and polynucleotide sequences encoding the DGATs and PDAT1 enzymes; polynucleotide constructs, vectors and host cells incorporating the polynucleotide sequences; and methods of producing and using same.

### BACKGROUND OF THE INVENTION

Pomegranate (Punica granatum) seed oil has been attracting increasing interest since its main component, punicic acid, may be used as a therapeutic agent in inflammatory diseases and as a dietary agent for chemoprevention of prostate and breast cancer (Kim et al., 2002; Shyed et al., 2008; Boussetta et al., 2009). Punicic acid (18:3Δ^{9cis, 11trans, 13cis}) is an uncommon form of conjugated linolenic acid. The conjugated double bond in punicic acid is synthesized by a divergent form of the Δ12-oleic acid desaturase (fatty acid conjugase, designated here as FADX) which catalyzes the conversion of the Δ12 double bond of linoleic acid (18:2Δ^{9cis, 12cis}) into two conjugated and trans-cis configurated double bonds at the 11 and 13 positions (Hornung et al., 2002; Iwabuchi et al., 2003). Delta-12 desaturase (PgFAD2, GenBank accession #AY178447) and fatty acid conjugase (PgFADX, GenBank accession #AY178446) involved in the synthesis of punicic acid in pomegranate have been isolated by PCR based cloning. Over-expression of PgFADX in Arabidopsis thaliana resulted in the limited accumulation of punicic acid up to 3.5% accompanied by increased accumulation of oleic acid (Iwabuchi et al., 2003; Cahoon et al., 2006). These amounts of punicic acid are considerably lower compared to the amounts in seeds of P. granatum (up to 80%).

Similar problems with low accumulation of other conjugated fatty acids in transgenic plants have been reported. Genes encoding divergent FAD2 enzymes have been expressed transgenically in A. thaliana and oilseed plants, but resulted in relatively low levels of accumulation of the unusual fatty acid, usually <20% compared to the 60-90% typically found in the source species (Broun and Somerville, 1997; Cahoon et al., 1999; Dyer and Mullen 2007; Dyer et al., 2008, van Erp et al., 2011). Oleic acid content increased considerably in these transgenic plants, suggesting that production of the unusual fatty acids in some way inhibits the FAD2 desaturase activity (Cahoon et al., 2006; Zhou et al., 2006; Thomaeus et al., 2001). This was ascribed to the competition between the housekeeping FAD2 and the diverged FAD2-like enzymes, and an inhibition of the normal FAD2 by the conjugated acyl residues in the phosphatidylcholine (PC) substrate molecules (Drexler et al., 2003).

It has been recently demonstrated that levels of unusual/conjugated fatty acids present on PC in transgenic plants were substantially higher than those observed in the source plants, indicating that accumulation of these fatty acids in transgenic plants is primarily limited by their inefficient removal from PC and passage through the Kennedy pathway (Cahoon et al., 2006; Cahoon et al., 2007; Dyer et al., 2008). The route to a high-level of accumulation of punicic acid in transgenic plants may necessitate the identification and introduction of genes encoding key enzymes such as phospholipases PLA1 and PLA2 (Stahl et al., 1995; Singh et al., 2005), diacylglycerol acyltransferase type 1 (DGAT1) and 2 (DGAT2) (Burgal et al., 2008; Li et al., 2010; Shockey et al., 2006), and phospholipid:diacylglycerol acyltransferase (PDAT) (van Erp et al., 2011). Over-expression of DGAT1, DGAT2 and PDAT led to increased levels of other unusual fatty acids such as epoxy and hydroxyl fatty acids in transgenic plants (Li et al., 2010; Burgal et al., 2008; van Erp et al., 2011).

### SUMMARY OF THE INVENTION

In general terms, the present invention relates to transgenic plants with enhanced punicic acid accumulation resulting from over-expression of PgFADX and PgFAD2. In one embodiment, the plant further expresses one or more of PgDGAT1, PgDGAT2, and PgPDAT1. The present invention also relates to isolated PgDGAT1, PgDGAT2, and PgPDAT1 genes from Punica granatum, and methods for their use.

In one aspect, the present invention comprises a method of increasing punicic acid production in an oilseed plant, comprising the step of transforming the plant to over-express PgFADX and PgFAD2. In one embodiment, the method further comprises the step of transforming the plant to over-express a DGAT and/or a PDAT or a gene encoding a DGAT and/or a PDAT Preferably, the encoded DGAT or PDAT comprises a PgDGAT or PgPDAT, such as PgDGAT1, PgDGAT2 and PgPDAT1.

The transgenic oilseed plant may comprise linseed, rapeseed, canola, peanut, safflower, flax, hemp, camelina, soybean, pea, sunflower, olive, palm, oats, wheat, triticale, barley, corn, thale cress, or legume. In one embodiment, the plant comprises Arabidopsis thaliana, which may be an Arabidopsis thaliana fad3/fae1 double mutant.

In another aspect, the invention may comprise a transgenic plant, plant cell, plant seed, callus, plant embryo, microspore-derived embryo, or microspore, comprising a recombinant expression vector encoding PgFADX and PgFAD2. In one embodiment, the transgenic plant, plant cell, plant seed, callus, plant embryo, microspore-derived embryo, or microspore further comprises an expression vector encoding one or more of a PgDGAT and PgPDAT, which may comprise one or more of PgDGAT1, PgDGAT2 or PgPDAT1.

In another aspect, the invention may consist or comprise an isolated polynucleotide sequence encoding a protein or polypeptide comprising or consisting of an amino acid sequence selected from SEQ ID NO: 2, 4 or 6, respective biologically active variants and biologically active portions thereof, with respective sequences having at least 85% identity thereto, and wherein the variants have diacylglycerol acyltransferase type 1 (DGAT1), type 2 (DGAT2) or phospholipid diacylglycerol acyltransferase (PDAT) activity.

In one embodiment, the polynucleotide encodes a polypeptide having DGAT activity and comprising the amino acid sequence of SEQ ID NO: 2 or 4, or an amino acid sequence having DGAT activity and having at least 85% sequence identity therewith, or a polypeptide having PDAT activity and comprising the amino acid sequence of SEQ ID NO: 6, or an amino acid sequence having PDAT activity and having at least 85% sequence identity therewith.

The isolated polynucleotide may consist or comprise the nucleotide sequence of SEQ ID NO: 1, 3 or 5.

Additional aspects and advantages of the present invention will be apparent in view of the description, which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The invention will now be described by way of an exemplary embodiment with reference to the accompanying simplified, diagrammatic, not-to-scale drawings. In the drawings:

FIG. 1 is a schematic diagram showing the pathway for synthesizing punicic acid.

FIG. 2 is a schematic diagram showing the NCJ and NCJD constructs used to transform A. thaliana fad3/fae1 plants.

FIG. 3 shows the fatty acid composition (i.e., proportions of 18:1, 18:2, and punicic acid contributing to the total fatty acid profile) in the seed oils of A. thaliana fad3/fae1 mutant-T₂ (Hi) plants transformed with the NCJ construct to over-express the PgFADX gene compared to non-transformed plants.

FIG. 4 shows the fatty acid composition (i.e., proportions of 18:1, 18:2, and punicic acid contributing to the total fatty acid profile) in the seed oils of A. thaliana fad3/fae1 mutant-T₂ (Hi) plants transformed with the NCJD construct to over-express the PgFADX and PgFAD2 genes compared to non-transformed plants.

FIG. 5 shows the fatty acid composition (i.e., proportions of 18:1, 18:2, and punicic acid contributing to the total fatty acid profile) in the seed oils of A. thaliana fad3/fae1 mutant-T₃ (Ho) plants transformed with the NCJ construct to over-express the PgFADX gene compared to non-transformed and null segregant plants.

FIG. 6 shows the fatty acid composition (i.e., proportions of 18:1, 18:2, and punicic acid within the total fatty acid profile) in the seed oils of A. thaliana fad3/fae1 mutant-T₃ (Ho) plants transformed with the NCJD construct to over-express the PgFADX and PgFAD2 genes compared to non-transformed plants.

FIG. 7 shows the fatty acid composition (i.e., proportions of 18:1, 18:2, and punicic acid contributing to the total fatty acid profile) in the seed oils of A. thaliana fad3/fae1 mutant-T₂ (Hi) plants transformed with the SAF4 construct to over-express the PgFADX, PgFAD2 and PgDGAT2 genes compared to non-transformed plants.

FIG. 8 shows the fatty acid composition (i.e., proportions of 18:1, 18:2, and punicic acid contributing to the total fatty acid profile) in the seed oils of A. thaliana fad3/fae1 mutant-T₃ (Ho) plants transformed with the SAF4 construct to over-express the PgFADX, PgFAD2 and PgDGAT2 genes or with the NCJD construct to over-express the PgFADX and PgFAD2 genes compared to non-transformed plants.

FIG. 9 shows the relative content of punicic acid in phosphatidylcholine (PC) and triacylglycerol (TAG) from P. granatum and A. thaliana seed line NCJD-30-2. The values represented by the bars are the average±SD from analyses of three independent samples.

FIG. 10A shows the PgDGAT1 nucleotide sequence, and FIG. 10B shows the PgDGAT1 amino acid sequence.

FIG. 11A shows the PgDGAT2 nucleotide sequence, and FIG. 11B shows the PgDGAT2 amino acid sequence.

FIG. 12A shows the PgPDAT1 nucleotide sequence, and FIG. 12B shows the PgPDAT1 amino acid sequence.

FIG. 13 shows the relative content of punicic acid in polar lipids (PL) and triacylglycerol (TAG) from yeast cells over-expressing PgFADX, PgFADX+PgDGAT1, and PgFADX+PgDGAT2. The values represented by the bars are the average±SD from analyses of three independent samples.

### DETAILED DESCRIPTION OF PREFERRED EMBODIMENTS

The present invention relates to transgenic plants with enhanced punicic acid accumulation resulting from over-expression of PgFADX and PgFAD2, and optionally, one or more of PgDGAT1, PgDGAT2, and PgPDAT1. The present invention also relates to isolated polynucleotides and polypeptides of the PgDGAT1, PgDGAT2, and PgPDAT1 genes from Punica granatum; nucleic acid constructs, recombinant expression vectors and host cells incorporating the polynucleotide sequences; and methods of producing and using same. When describing the present invention, all terms not defined herein have their common art-recognized meanings. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims.

To facilitate understanding of the invention, the following definitions are provided.

A “cDNA” is a polynucleotide which is complementary to a molecule of mRNA. The “cDNA” is formed of a coding sequence flanked by 5′ and 3′ untranslated sequences.

A “coding sequence” or “coding region” or “open reading frame (ORF)” is part of a gene that codes for an amino acid sequence of a polypeptide.

A “complementary sequence” is a sequence of nucleotides which forms a duplex with another sequence of nucleotides according to Watson-Crick base pairing rules where “A” pairs with “T” and “C” pairs with “G.” For example, for the polynucleotide 5′-AATGCCTA-3′ the complementary sequence is 5′-TAGGCATT-3′.

A “construct” is a polynucleotide which is formed by polynucleotide segments isolated from a naturally occurring gene or which is chemically synthesized. The “construct” which is combined in a manner that otherwise would not exist in nature, is usually made to achieve certain purposes. For instance, the coding region from “gene A” can be combined with an inducible promoter from “gene B” so the expression of the recombinant construct can be induced.

“Downstream” means on the 3′ side of a polynucleotide while “upstream” means on the 5′ side of a polynucleotide.

“Expression” refers to the transcription of a gene into RNA (rRNA, tRNA) or messenger RNA (mRNA) with subsequent translation into a protein.

“Gene” means a DNA segment which contributes to phenotype or function, and which may be characterized by sequence, transcription or homology.

“Isolated” means that a substance or a group of substances is removed from the coexisting materials of its natural state.

“Nucleic acid” means polynucleotides such as deoxyribonucleic acid (DNA), and, where appropriate, ribonucleic acid (RNA). The term should also be understood to include, as equivalents, analogs of either RNA or DNA.

“Plasmid” means a DNA molecule which is separate from, and can replicate independently of, the chromosomal DNA. They are double stranded and, in many cases, circular. Plasmids used in genetic engineering are known as vectors and are used to multiply or express particular genes. Any plasmid may be used for the present invention provided that the plasmid contains a gene which encodes a PgDGAT1, PgDGAT2, and PgPDAT1, or a variant thereof in an expressible manner. In one embodiment, the plasmid comprises a yeast expression vector. Those skilled in art will recognize that any plasmid in the art may be modified for use in the compositions and methods of the present invention.

“Regulatory element” includes, but is not limited to, a promoter, enhancer, terminator, and the like which are required for the expression of the encoded PgDGAT1, PgDGAT2, and PgPDAT1, or variant thereof.

A “polynucleotide” is a linear sequence of ribonucleotides (RNA) or deoxyribonucleotides (DNA) in which the 3′ carbon of the pentose sugar of one nucleotide is linked to the 5′ carbon of the pentose sugar of another nucleotide. The deoxyribonucleotide bases are abbreviated as “A” deoxyadenine; “C” deoxycytidine; “G” deoxyguanine; “T” deoxythymidine; “I” deoxyinosine. Some oligonucleotides described herein are produced synthetically and contain different deoxyribonucleotides occupying the same position in the sequence, The blends of deoxyribonucleotides are abbreviated as “W” A or T; “Y” C or T; “H” A, C or T; “K” G or T; “D” A, G or T; “B” C, G or T; “N” A, C, G or T.

A “polypeptide” is a linear sequence of amino acids linked by peptide bonds. Common amino acids are abbreviated as “A” alanine; “R” arginine; “N” asparagine; “D” aspartic acid; “C” cysteine; “Q” glutamine; “E” glutamic acid; “G” glycine; “H” histidine; “I” isoleucine; “L” leucine; “K” lysine; “M” methionine; “F” phenylalanine; “P” proline; “S” serine; “T” threonine; “W” tryptophan; “Y” tyrosine and “V” valine.

Two polynucleotides or polypeptides are “identical” if the sequence of nucleotides or amino acids, respectively, in the two sequences is the same when aligned for maximum correspondence as described here. Sequence comparisons between two or more polynucleotides or polypeptides can be generally performed by comparing portions of the two sequences over a comparison window which can be from about 20 to about 200 nucleotides or amino acids, or more. The “percentage of sequence identity” may be determined by comparing two optimally aligned sequences over a comparison window, wherein the portion of a polynucleotide or a polypeptide sequence may include additions (i.e., insertions) or deletions (i.e., gaps) as compared to the reference sequence. The percentage is calculated by determining the positions at which identical nucleotides or identical amino acids are present, dividing by the number of positions in the window and multiplying the result by 100 to yield the percentage of sequence identity. Polynucleotide and polypeptide sequence alignment may be performed by implementing specialized algorithms or by inspection. Examples of sequence comparison and multiple sequence alignment algorithms are: BLAST and ClustalW software. Identity between nucleotide sequences can also be determined by DNA hybridization analysis, wherein the stability of the double-stranded DNA hybrid is dependent on the extent of base pairing that occurs. Conditions of high temperature and/or low salt content reduce the stability of the hybrid, and can be varied to prevent annealing of sequences having less than a selected degree of homology. Hybridization methods are described in Ausubel et al. (2000).

An “oleic acid” is a monounsaturated omega-9 fatty acid which is abbreviated with a lipid number of 18:1 cis-9.

A “linoleic acid” is an unsaturated omega-6 fatty acid which is abundant in many vegetable oils, and is an essential dietary requirement for all mammals lacking the delta-12 desaturase involved in its synthesis.

A “punicic acid” is a conjugated linolenic acid isomer containing cis-Δ9, trans-Δ11, cis-Δ13 double bonds in the C₁₈ carbon chain and having the structure below:

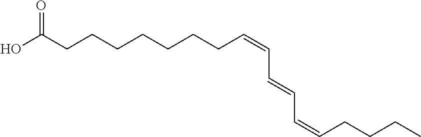 [SMILES: CCCC\C=C/C=C/C=C\CCCCCCCC(=O)O]

A “fatty acid conjugase” (FADX) is an enzyme which utilizes linoleic acid as a substrate for punicic acid synthesis. A Punica granatum fatty acid conjugase (PgFADX) is an enzyme which catalyzes the conversion of the delta-12 double bond of linoleic acid (18:2Δ^{9cis, 12cis}) into two conjugated and trans-cis configurated double bonds at the 11 and 13 positions. A “PgFADX” refers to a polypeptide from Punica granatum which exhibits FADX enzymatic activity. A polypeptide having “FADX activity” is a polypeptide that has, to a greater or lesser degree, the enzymatic activity of FADX.

A “PgFADX” is a gene encoding a FADX from Punica granatum.

A “PgFAD2” is a gene encoding a FAD2 from Punica granatum (pomegranate).

A “PgFAD2” refers to a polypeptide from Punica granatum which exhibits FAD2 enzymatic activity (delta-12 desaturase). A polypeptide having “FAD2 activity” is a polypeptide that has, to a greater or lesser degree, the enzymatic activity of FAD2.

A “triacylglycerol” is an ester having three fatty carboxylic acids attached to a single glycerol backbone. It is the main component of vegetable oil and animal fats. Alternative names include triglyceride or triacylglyceride, abbreviated TG or TAG.

A diacylglycerol acyl transferase (DGAT) is an enzyme of the class EC 2.3.1.20 which catalyzes the reaction: acyl-CoA+sn-1,2-diacylglycerol→CoA+triacylglycerol. Alternative names include: diacylglycerol O-acyltransferase, diacylglycerol acyltransferase, diglyceride acyltransferase and acylCoA:diacylglycerol acyltransferase. A polypeptide having “DGAT activity” is a polypeptide that has, to a greater or lesser degree, the enzymatic activity of DGAT.

A “PgDGAT” is a gene encoding a DGAT from Punica granatum. Two types of P. granatum DGATs are described here: type 1 (DGAT1) and type 2 (DGAT2).

A “PgDGAT” refers to a polypeptide from Punica granatum which exhibits DGAT enzymatic activity. Two types of PgDGATs are described here (for example, type 1: PgDGAT1 or type 2: PgDGAT2). A number denoted after PgDGAT refers to a specific polypeptide which exhibits DGAT enzyme activity.

A “phospholipid:diacylglycerol acyl transferase” (PDAT) is an enzyme of the class EC 2.3.1.158 which catalyzes the reaction: phospholipid+1,2-diacylglycerol⇄ lysophospholipid+TAG. A polypeptide having “PDAT activity” is a polypeptide that has, to a greater or lesser degree, the enzymatic activity of PDAT.

A “PgPDAT1” is a gene encoding a PDAT from Punica granatum. A number denoted after PgPDAT (for example, PgPDAT1) refers to a specific gene encoding a PDAT.

A “PgPDAT1” refers to a polypeptide from Punica granatum which exhibits PDAT enzymatic activity. A number denoted after PgPDAT (for example, PgPDAT1) refers to a specific polypeptide which exhibits PDAT enzyme activity.

A “promoter” is a polynucleotide usually located within 20 to 5000 nucleotides upstream of the initiation of translation site of a gene. The “promoter” determines the first step of expression by providing a binding site to DNA polymerase to initiate the transcription of a gene. The promoter is said to be “inducible” when the initiation of transcription occurs only when a specific agent or chemical substance is presented to the cell. For instance, the GAL “promoter” from yeast is “inducible by galactose,” meaning that this GAL promoter allows initiation of transcription and subsequent expression only when galactose is presented to yeast cells.

“Transformation” means the directed modification of the genome of a cell by external application of a polynucleotide, for instance, a construct. The inserted polynucleotide may or may not integrate with the host cell chromosome. For example, in bacteria, the inserted polynucleotide usually does not integrate with the bacterial genome and might replicate autonomously. In plants, the inserted polynucleotide integrates with the plant chromosome and replicates together with the plant chromatin.

A “transgenic” organism is the organism that was transformed with an external polynucleotide. The “transgenic” organism encompasses all descendants, hybrids and crosses thereof, whether reproduced sexually or asexually and which continue to harbor the foreign polynucleotide.

A “vector” is a polynucleotide that is able to replicate autonomously in a host cell and is able to accept other polynucleotides. For autonomous replication, the vector contains an “origin of replication.” The vector usually contains a “selectable marker” that confers the host cell resistance to certain environment and growth conditions. For instance, a vector that is used to transform bacteria usually contains a certain antibiotic “selectable marker” which confers the transformed bacteria resistance to such antibiotic.

The present invention relates to a method for increasing the production of punicic acid in oilseed plants through the over-expression of PgFADX and PgFAD2, and optionally, one or more of PgDGAT1, PgDGAT2, and PgPDAT1, and a method for the production of oils having an increased content of punicic acid. The invention furthermore relates to the production of transgenic plants, preferably a transgenic oilseed plant, having an increased content of punicic acid. The present invention also relates to isolated polynucleotides and polypeptides of the PgDGAT1, PgDGAT2, and PgPDAT1 genes from Punka granatum; nucleic acid constructs, vectors and host cells incorporating the polynucleotide sequences; and methods of producing and using same.

In one aspect, the invention provides isolated PgDGAT1, PgDGAT2, and PgPDAT1 polynucleotides, and polypeptides having DGAT or PDAT activity. PgDGAT1, PgDGAT2, and PgPDAT1 polynucleotides include, without limitation (1) single- or double-stranded DNA, such as cDNA or genomic DNA including sense and antisense strands; and (2) RNA, such as mRNA. PgDGAT1, PgDGAT2, and PgPDAT1 polynucleotides include at least a coding sequence which codes for the amino acid sequence of the specified PgDGAT1, PgDGAT2 and PgPDAT1 polypeptide, but may also include 5′ or 3′ untranslated regions and transcriptional regulatory elements such as promoters and enhancers found upstream or downstream from the transcribed region.

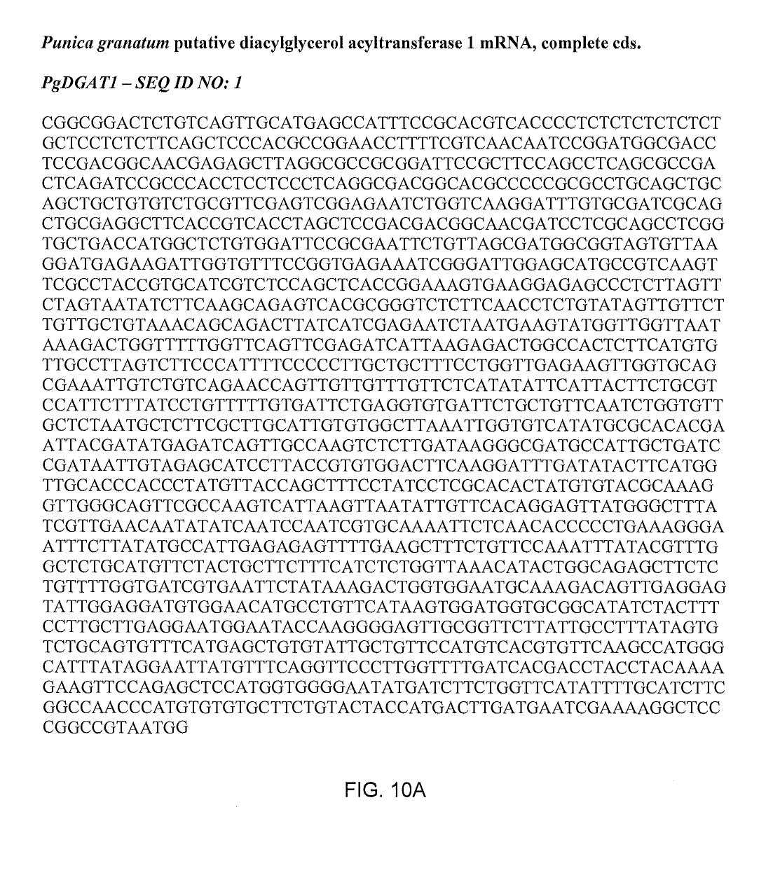

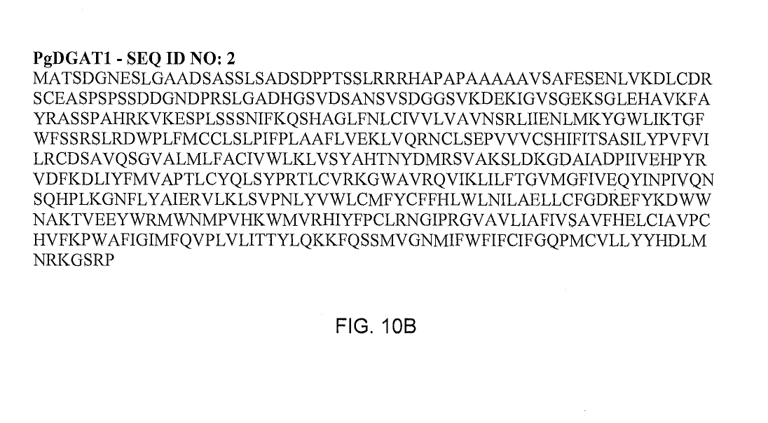

In one embodiment, the invention provides a PgDGAT1 polynucleotide which is a cDNA comprising the nucleotide sequence of 1732 base pairs depicted in SEQ ID NO: 1 (FIG. 10A), and which was isolated from Punica granatum. The cDNA comprises a coding region of 1623 base pairs (107-1729 region/position of SEQ ID NO: 1). The PgDGAT1 encoded by the coding region (SEQ ID NO: 2; FIG. 10B) is a 540 amino acid polypeptide.

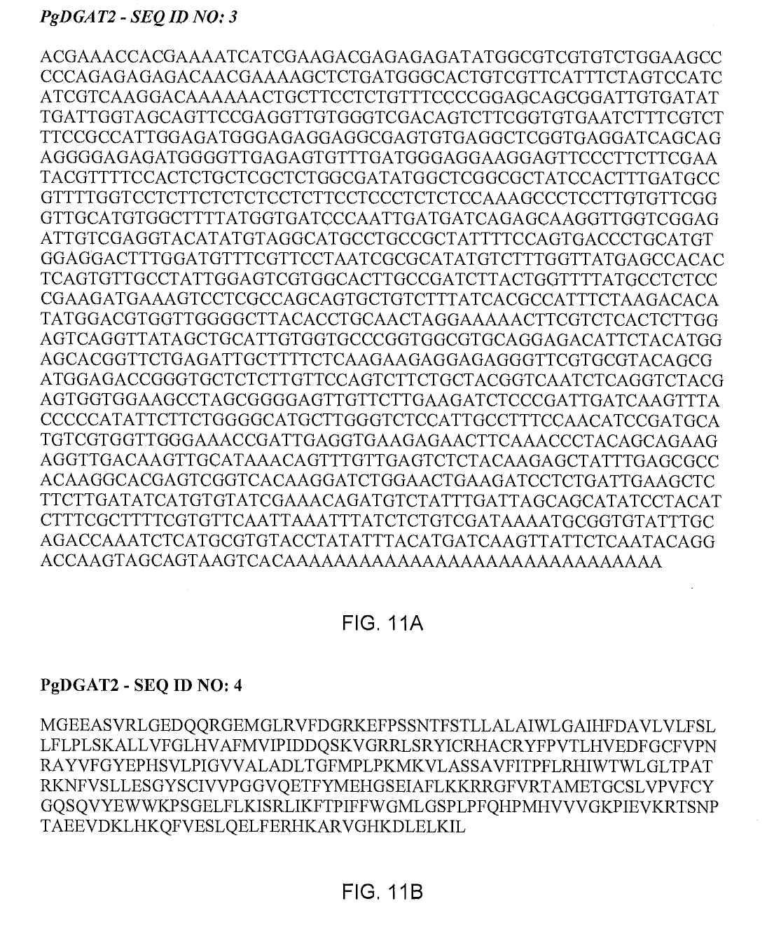

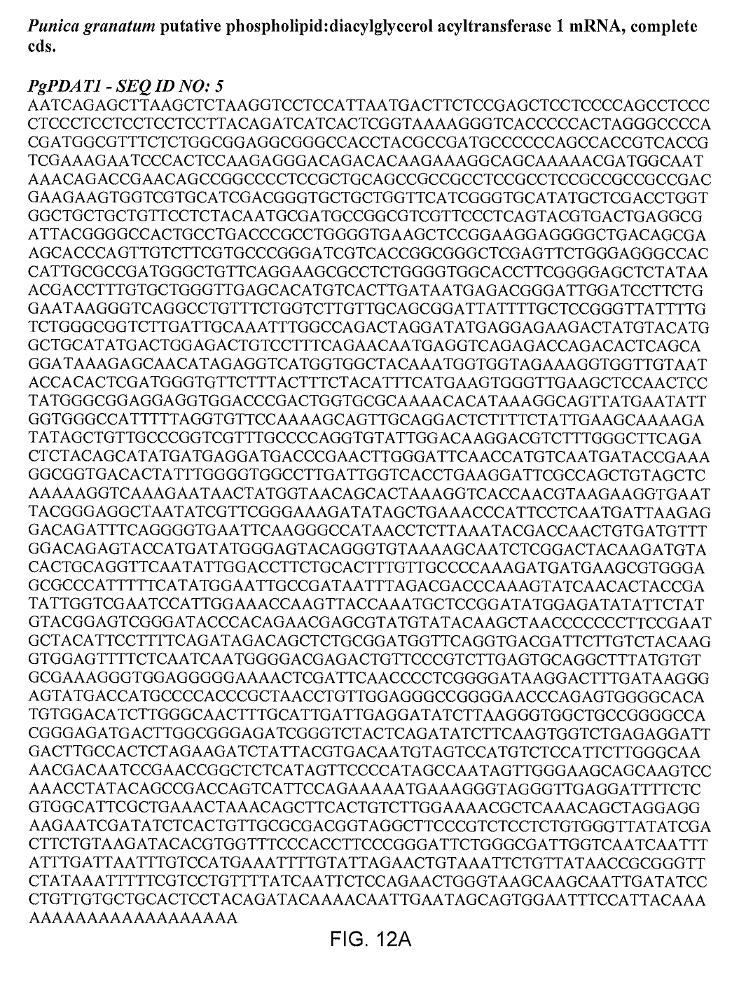

In one embodiment, the invention provides a PgDGAT2 polynucleotide which is a cDNA comprising the nucleotide sequence of 1479 base pairs depicted in SEQ ID NO: 3 (FIG. 11A), and which was isolated from Punica granatum. The cDNA comprises a coding region of 1005 base pairs (243-1247 region/position of SEQ ID NO: 3). The PgDGAT2 encoded by the coding region (SEQ ID NO: 4; FIG. 11B) is a 334 amino acid polypeptide.

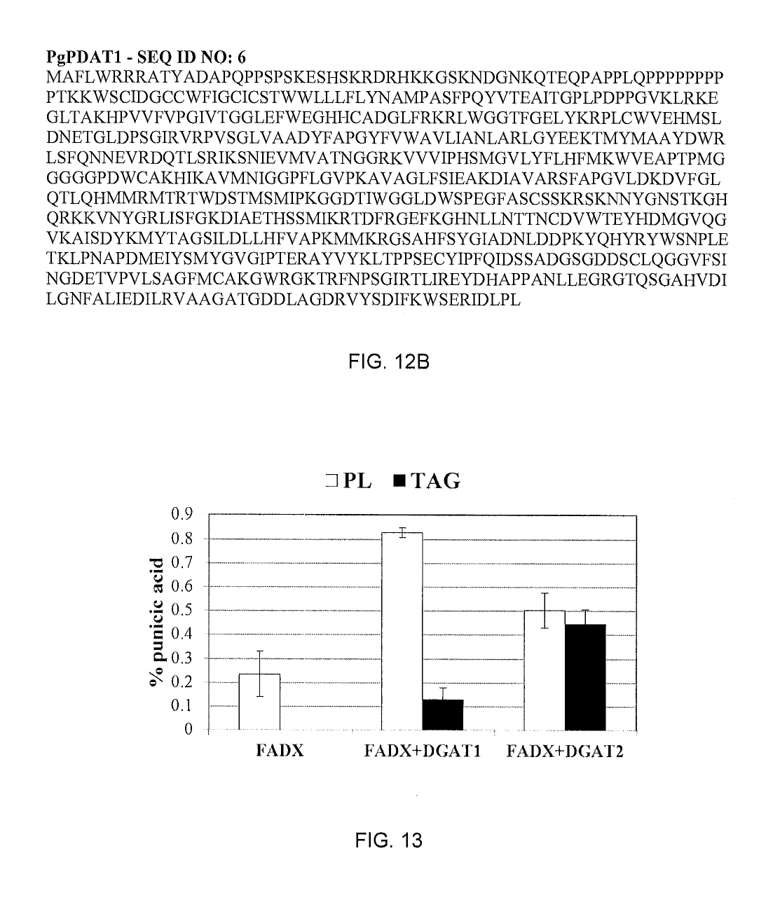

In one embodiment, the invention provides a PgPDAT1 polynucleotide which is a cDNA comprising the nucleotide sequence of 2743 base pairs depicted in SEQ ID NO: 5 (FIG. 12A), and which was isolated from Punica granatum. The cDNA comprises a coding region of 2052 base pairs (129-2180 region/position of SEQ ID NO: 5). The PgPDAT1 encoded by the coding region (SEQ ID NO: 6; FIG. 12B) is a 683 amino acid polypeptide.

Those skilled in the art will recognize that the degeneracy of the genetic code allows for a plurality of polynucleotides to encode for identical polypeptides. Accordingly, the invention includes polynucleotides of SEQ ID NOS: 1, 3, and 5, and variants of polynucleotides encoding the polypeptides of SEQ ID NOS: 2, 4 and 6. In one embodiment, polynucleotides having at least 85%, at least 90%, at least 95%, at least 96%, at least 97%, at least 98%, or at least 99% sequence identity to the nucleotide sequences depicted in SEQ ID NOS: 1, 3, and 5 are included in the invention. Methods for isolation of such polynucleotides are well known in the art (see for example, Ausubel et al., 2000).

In one embodiment, the invention provides isolated polynucleotides which encode PgDGAT1, PgDGAT2, and PgPDAT1, or polypeptides having amino acid sequences having at least 85%, at least 90%, at least 95%, at least 96%, at least 97%, at least 98%, or at least 99% sequence identity to the amino acid sequences depicted in SEQ ID NOS: 2, 4, and 6.

The above described polynucleotides of the invention may be used to express polypeptides in recombinantly engineered cells including, for example, bacterial, yeast, fungal, mammalian or plant cells. In one embodiment, the invention provides polynucleotide constructs, vectors and cells comprising PgDGAT1, PgDGAT2, and PgPDAT1 polynucleotides. Those skilled in the art are knowledgeable in the numerous systems available for expression of a polynucleotide. All systems employ a similar approach, whereby an expression construct is assembled to include the protein coding sequence of interest and control sequences such as promoters, enhancers, and terminators, with signal sequences and selectable markers included if desired. Briefly, the expression of isolated polynucleotides encoding polypeptides is typically achieved by operably linking, for example, the DNA or cDNA to a constitutive or inducible promoter, followed by incorporation into an expression vector. The vectors can be suitable for replication and integration in either prokaryotes or eukaryotes. Typical expression vectors include transcription and translation terminators, initiation sequences, and promoters useful for regulation of the expression of the DNA. High level expression of a cloned gene is obtained by constructing expression vectors which contain a strong promoter to direct transcription, a ribosome binding site for translational initiation, and a transcription/translation terminator. Vectors may further comprise transit and targeting sequences, selectable markers, enhancers or operators. Means for preparing vectors are well known in the art. Typical vectors useful for expression of polynucleotides in plants include for example, vectors derived from the Ti plasmid of Agrobacterium tumefaciens and the pCaM-VCN transfer control vector. Promoters suitable for plant cells include for example, the nopaline synthase, octopine synthase, and mannopine synthase promoters, and the caulimovirus promoters. Seed-specific promoters, such as ACP and napin-derived transcription initiation regions, have been shown to confer preferential expression of a specific gene in plant seed tissue. In one embodiment, the seed-specific napin promoter is preferred.

Those skilled in the art will appreciate that modifications (i.e., amino acid substitutions, additions, deletions and post-translational modifications) can be made to a polypeptide of the invention without eliminating or diminishing its biological activity. Conservative amino acid substitutions (i.e., substitution of one amino acid for another amino acid of similar size, charge, polarity and conformation) or substitution of one amino acid for another within the same group (i.e., nonpolar group, polar group, positively charged group, negatively charged group) are unlikely to alter protein function adversely. Some modifications may be made to facilitate the cloning, expression or purification, Variant PgDGAT1, PgDGAT2, and PgPDAT1 polypeptides may be obtained by mutagenesis of the corresponding polynucleotides depicted in SEQ ID NOS: 1, 3 and 5 using techniques known in the art including, for example, oligonucleotide-directed mutagenesis, region-specific mutagenesis, linker-scanning mutagenesis, and site-directed mutagenesis by PCR (Ausubel et al., 2000).

Various methods for transformation or transfection of cells are available. For prokaryotes, lower eukaryotes and animal cells, such methods include for example, calcium phosphate precipitation, fusion of the recipient cells with bacterial protoplasts containing the DNA, treatment of the recipient cells with liposomes containing the DNA, DEAE dextran, electroporation, biolistics and microinjection. The transfected cells are cultured, and the produced PgDGAT1, PgDGAT2, and PgPDAT1 polypeptides may be isolated and purified from the cells using standard techniques known in the art. Various industrial strains of microorganisms including for example, fungi, such as Mortierella or Traustochytrium; mosses such as Physcomitrella or Ceratodon; algae such as Crypthecodinium or Phaeodactylum; or Aspergillus, Pichia pastoris, Saccharomyces cerevisiae may be used to produce PgDGAT1, PgDGAT2, and PgPDAT1 polypeptides. Alternatively, exogenous DNA may be transferred into yeast by electroporation, biolistics, glass bead agitation and spheroplasts.

Methods for transformation of plant cells include for example, infiltration, electroporation, PEG poration, particle bombardment, Agrobacterium tumefaciens- or Agrobacterium rhizogenes-mediated transformation, direct protoplast transformation, and microinjection. The transformed plant cells, seeds, callus, embryos, microspore-derived embryos, microspores, organs or explants are cultured or cultivated using standard plant tissue culture techniques and growth media to regenerate a whole transgenic plant which possesses the transformed genotype. Transformation may be confirmed by use of a DNA marker gene encoding for an enzyme that confers herbicide tolerance or antibiotic resistance; catalyzes deamination of D-amino acids; or by conducting methods such as PCR or Southern blot hybridization. Transgenic plants may pass polynucleotides encoding desired polypeptides to their progeny, or can be further crossbred with other species. Accordingly, in one embodiment, the invention provides methods for producing transgenic plants, plant cells, callus, seeds, plant embryos, microspore-derived embryos, and microspores comprising PgFADX and PgFAD2, and optionally, one or more of PgDGAT1, PgDGAT2, and PgPDAT1 polynucleotides.

In one embodiment, the invention provides transgenic plants, plant cells, callus, seeds, plant embryos, microspore-derived embryos, or microspores, comprising PgFADX and PgFAD2 polynucleotides, and optionally, one or more of PgDGAT1, PgDGAT2, and PgPDAT1 polynucleotides. Plant species of interest for transformation include, without limitation, oilseeds (for example, the linseed plant, rapeseed or canola, peanut, safflower), flax, hemp, camelina, canola, sunflower, olive, palm, oats, wheat, triticale, barley, corn, thale cress, and legume plants including soybean and pea. In one embodiment, the plant comprises Arabidopsis thaliana. In one embodiment, the plant comprises Arabidopsis thaliana fad3/fae1 double mutant.

In one embodiment, the invention comprises a method of increasing punicic acid production in an oilseed plant, comprising the step of transforming a host cell under conditions sufficient for over-expression of PgFADX and PgFAD2, and generating a transgenic plant from the host cell. In one embodiment, the host cell is also transformed to over-express one or more of a DGAT or PDAT.

The following describes specific examples of embodiments of the present invention. It will be appreciated by those skilled in the art that the isolated polynucleotide and polypeptides of the PgDGAT1, PgDGAT2, and PgPDAT1 genes from Punica granatum have industrial and nutritional applications. The PgDGAT1, PgDGAT2, and PgPDAT1 genes encode PgDGAT1, PgDGAT2, and PgPDAT1, respectively. The DGAT and PDAT polynucleotides and polypeptides may be used in the industrial production of punicic acid using recombinant technology using transformed bacterial, yeast or fungal cells. Transformed cells may be engineered to accumulate punicic acid which may be incorporated into human food and animal feed applications to produce health supplements or to improve the nutritional quality of products. These examples demonstrate how these genes can be used to produce punicic acid in transgenic cells and plants.

The A. thaliana fad3/fae1 double mutant is deficient in delta-15-desaturase (FAD3) and fatty acid elongase (FAE1) activity. A. thaliana FAD3 catalyzes the conversion of linoleic acid (18:2Δ^{9, 12}) into linolenic (8:3Δ^{9, 12, 15}), while FAE1 catalyzes the elongation of oleic acid (18:1Δ⁹) mostly into eicosenoic acid (20:1Δ¹¹). Since oleic acid is a substrate for ongoing desaturation and elongation, consequently, the A. thaliana fad3/fae1 double mutant has a two-fold higher amount of linoleic acid substrate than is present in a wild type A. thaliana, which may lead to a significant increase of punicic acid accumulation in the seed oil. However, since substrate availability limits punicic acid production in transgenic A. thaliana seeds, additional genes and enzymes may enhance punicic acid accumulation.

As described in the following Examples, the A. thaliana fad3/fae1 double mutant was transformed with various expression constructs comprising PgFADX and PgFAD2, as well as with PgDGAT2. Seeds from the treated plants were plated out, with the transformants being selected and transferred to soil to establish primary transgenic plants (T₁) which were grown to maturity. T₂ seeds were harvested from the T₁ plants, analyzed for the fatty acid composition, and used to establish T₂ progeny plants. T₃ seeds were harvested and analyzed for the fatty acid composition.

PgFADX catalyzes the conversion of the delta-12 double bond of linoleic acid into two conjugated and trans-cis configurated double bonds at the 11 and 13 positions. PgFADX is a fatty acid conjugase which utilizes linoleic acid as a substrate for punicic acid synthesis. Over-expression of both PgFADX and PgFAD2 genes in the transformed A. thaliana fad3/fae1 double mutant restored the relative proportion of C18:1 fatty acids to normal levels and resulted in a two-fold increase in the punicic acid content compared to that which was present in plants over-expressing only PgFADX.

Punicic acid is synthesized in developing seed tissues and accumulates in the seed storage lipid in the form of triacylglycerol (TAG). TAGs may be synthesized through a combination of DGAT and PDAT activities. DGAT catalyzes the acyl-CoA-dependent synthesis of TAG, whereas PDAT catalyzes the transfer of a fatty acyl chain from sn-2 position of phosphatidylcholine (PC) to the sn-3 position of diacylglycerol DAG, producing TAG. Without being bound by theory, over-expression of PgDGAT1, PgDGAT2, and/or PgPDAT1 to produce PgDGAT1, PgDGAT2, and/or PgPDAT1 may facilitate the incorporation of punicic acid into TAGs, further enhancing the punicic acid accumulation in transformed plants carrying both PgFADX and PgFAD2. Over-expression of PgDGAT2 increased the punicic acid content compared to that which was present in plants over-expressing only PgFADX and PgFAD2. The combined over-expression of PgFADX, PgFAD2 and PgDGAT2 yielded the highest overall production of punicic acid in transformed A. thaliana seeds.

Exemplary embodiments of the present invention are described in the following Examples, which are set forth to aid in the understanding of the invention, and should not be construed to limit in any way the scope of the invention as defined in the claims which follow thereafter. As will be apparent to those skilled in the art, various modifications, adaptations and variations of the specific disclosure herein can be made without departing from the scope of the invention claimed herein.

### Example 1

### Isolation and Characterization of Fatty Acid Conjugase (PgFADX) and Delta-12 Desaturase (PgFAD2) from Punica granatum

Using sequence information available at GenBank (NCBI), PgFADX (1188 bp) and PgFAD2 (1164 bp) ORFs were amplified by PCR and cloned into pCR4-TOPO (Invitrogen). Sequence analysis confirmed that isolated PgFADX and PgFAD2 were identical with sequences submitted by Iwabuchi et al., 2003 (AY178446 and AY178447, respectively).

To confirm the function of PgFADX, the coding region was amplified by PCR with primers 5′-GAAATGGGAGCTGATGGAACAAT-3′ (forward F1, SEQ ID NO: 7) and 5′-TCAGAACTTGCTCTTGAACC-3′ (reverse R1, SEQ ID NO: 8); cloned under the control of GAL1 inducible promoter into pYES2.1/V5-His-TOPO yeast expression vector (Invitrogen); and expressed in Saccharomyces cerevisiae strain INVSc1 (Invitrogen).

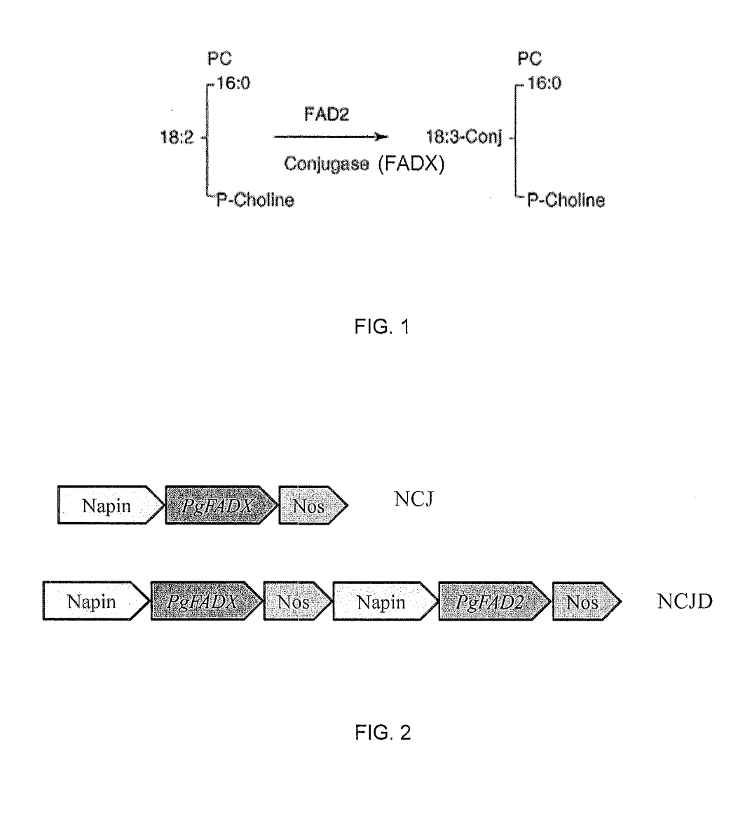

Punicic acid was detected (up to 1.2%) only in yeast cells expressing PgFADX after supplementation of the growth media with linoleic acid (18:2Δ^{9, 12}) at the final concentration of 300 μM. The results indicated that isolated PgFADX encodes a functional fatty acid conjugase utilizing linoleic acid as a substrate for punicic acid synthesis (FIG. 1).

### Example 2

### Preparation of Plant Transformation Vectors

The binary constructs NCJ and NCJD carrying the cassettes described below were developed in pRD400 vector background (CLONTECH) (FIG. 2).

For NCJ (napin-P/PgFADX/NOS-T), PCR was used to amplify the napin promoter (Josefsson et al., 1987), PgFADX, and Nos terminator (Bevan, 1983) using the primers set out in Table 1. The napin-P/PgFADX/NOS-T expression cassette was cloned in EcoRI and KpnI sites of pRD400 to yield the NCJ construct.

TABLE 1

| | | |
|---|---|---|
| napin | forward F2, EcoRI | 5′-ATAGAATTCAAGCTTTCTTCATCGGTGAT-3′ |
| promoter | site is underlined | (SEQ ID NO: 9) |
| | reverse R2, SmaI | 5′-ATACCCGGGGTCCGTGTATGTTTTTAATC-3′ |
| | site is underlined | (SEQ ID NO: 10) |
| PgFADX | forward F3, SmaI | 5′-TATCCCGGGATGGGAGCTGATGGAACA-3′ |
| | is underlined | (SEQ ID NO: 11) |
| | reverse R3, NotI | 5′-CGCGCGGCCGCTCAGAACTTGCTCTTGAAC-3′ |
| | site is underlined | (SEQ ID NO: 12) |
| Nos | forward primer F4, | 5′-CGCCGGCGGCCGCGATCGTTCAAACATTTGGCA-3′ |
| terminator | NotI site is | (SEQ ID NO: 13) |
| | underlined | |
| | reverse primer R4, | 5′-TATGGTACCCGATCTAGTAACATAGATGAC-3′ |
| | KpnI site is | (SEQ ID NO: 14) |
| | underlined | |

For NCJD (napin-P/PgFADX/NOS-T/Napin-P/PgFAD2/NOS-T), PCR was used to amplify the napin promoter, PgFAD2, and NOS terminator using the primers set out in Table 2. The napin-P/PgFAD2/NOS-T expression cassette was cloned into KpnI and Sail sites of NCJ (described above) to yield the NCJD construct.

TABLE 2

| | | |
|---|---|---|
| napin | forward F5, KpnI | 5′-ATAGGTACCAAGCTTTCTTCATCGGTGAT-3′ |
| promoter | site is underlined | (SEQ ID NO: 15) |
| | reverse R5, XhoI | 5′-ATACTCGAGGTCCGTGTATGTTTTTAATCT-3′ |
| | site is underlined | (SEQ ID NO: 16) |
| PgFAD2 | forward F6, XhoI | 5′-TAACTCGAGATGGGAGCCGGTGGAAG-3′ |
| | is underlined | (SEQ ID NO: 17) |
| | reverse R6, XbaI | 5′-TATTCTAGATCAGAGGTTCTTCTTGTAC-3′ |
| | site is underlined | (SEQ ID NO: 18) |
| Nos | forward F7, XbaI | 5′-TATTCTAGAGATCGTTCAAACATTTGGCAA-3′ |
| terminator | site is underlined | (SEQ ID NO: 19) |
| | reverse R7, SalI | 5′-ATAGTCGACCGATCTAGTAACATAGATGAC-3′ |
| | site is underlined | (SEQ ID NO: 20) |

For SAF4 (napin-P/PgDGAT2/NOS-T+napin-P/FADX/NOS-T+napin-P/PgFAD2/NOS-T), the napin-P/PgFADX/NOS-T expression cassette was amplified by PCR with primers F14: 5′-ATAGGCGCGCCAAGCTTTCTTCATCGGTGAT-3′ (SEQ ID NO: 21; AscI site is underlined) and R14: 5′-TAT GGTACCCGATCTAGTAACATAGATGAC-3′ (SEQ ID NO: 22; KpnI is underlined) using the NCJ plasmid as a template. The napin-P/PgFAD2/NOS-T expression cassette was amplified by PCR with primers F15: ATA GGTACCAAGCTTTCTTCATCGGTGAT-3′ (SEQ ID NO: 23; KpnI is underlined) and R15: TAT GGCGCGCCCGATCTAGTAACATAGATGAC-3′ (SEQ ID NO: 24; AscI is underlined) using the NCJD plasmid as a template. Both PCR products digested with respective restriction enzymes were ligated into AscI site of the binary vector pPZP-RCS1 (Goderis et al., 2002) resulting in the binary vector SAF2.

For the napin-P/PgDGAT2/NOS expression cassette, PCR was used to amplify the napin promoter, PgDGAT2, and NOS terminator using the primers set out in Table 3.

TABLE 3

| | | |
|---|---|---|
| napin | forward F16, AgeI | 5′-ATAACCGGTAAGCTTTCTTCATCGGTGAT-3′ |
| promoter | site is underlined | (SEQ ID NO: 25) |
| | reverse R16, XhoI | 5′-ATACTCGAGGTCCGTGTATGTTTTTAATCT-3′ |
| | site is underlined | (SEQ ID NO: 26) |
| PgDGAT2 | forward F17, XhoI | 5′-TAACTCGAGATGGGAGAGGAGGCGAG-3′ |
| | is underlined | (SEQ ID NO: 27) |
| | reverse R17, XbaI | 5′-ATATTCTAGATCAGAGGATCTTCAGTTCC-3′ |
| | site is underlined | (SEQ ID NO: 28) |
| Nos | forward F18, XbaI | 5′-TATTCTAGAGATCGTTCAAACATTTGGCAA-3′ |
| terminator | site is underlined | (SEQ ID NO: 29) |
| | reverse R18, NotI | 5′-CGCGCGGCCGCCGATCTAGTAACATAGATGAC-3′ |
| | site is underlined | (SEQ ID NO: 30) |

The napin-P/PgDGAT2/NOS-T expression cassette was cloned in AgeI and NotI sites of pAUX3131 (Goderies et al., 2002) resulting in ND2X31 plasmid. The napin-P/PgDGAT2/NOS-T cassette was then cut out from ND2X31 with I-SceI restriction enzyme and cloned into the corresponding site of SAF2 to yield the SAF4 construct.

### Example 3

### Production of Punicic Acid in A. thaliana Seeds

The binary vectors NCJ or NCJD were electroporated into Agrobacterium tumefaciens cell strain GV3101, while SAF4 was electroporated into Agrobacterium tumefaciens cell strain EHA101. The vectors were introduced into Arabidopsis thaliana fad3/fae1 (Smith et al., 2003) double mutant background using the floral dip method (Clough and Bent, 1998). Transgenic plants were selected and analyzed as described before (Mietkiewska et al., 2007).

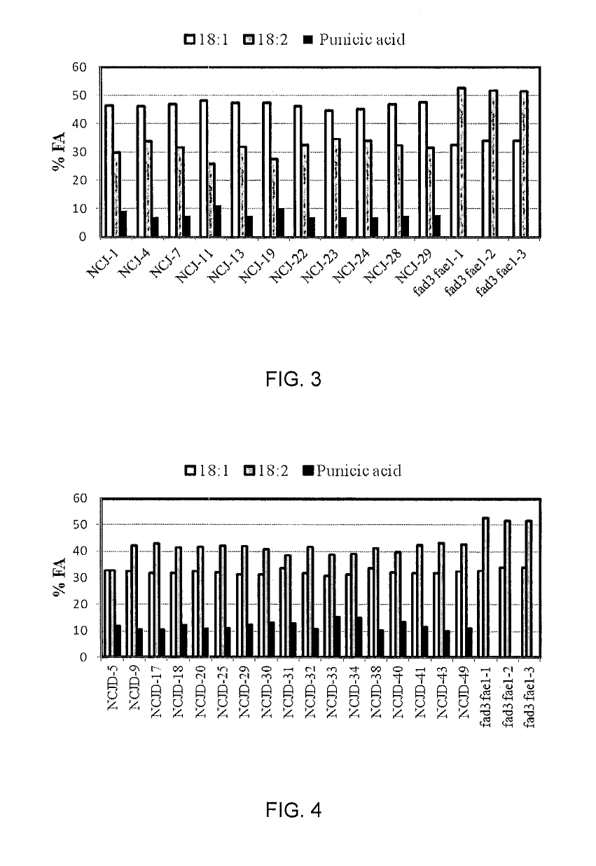

Seeds from twenty-eight independent transgenic lines carrying NCJ construct in high linoleic acid (52%) A. thaliana fad3/fae1 mutant background were analyzed. Results from the best 11 A. thalina T₂ lines are shown in FIG. 3. Significant changes in fatty acid composition in comparison to the control lines (fad3/fae1) were found. Seed specific expression of PgFADX resulted in an increased proportion of punicic acid (18:3Δ^{9Z,11E,13Z}) from 0% in the controls up to 11.26% in the best transgenic line NCJ-11. The increased proportion of punicic acid was correlated with concomitant reduction in the proportion of its corresponding precursor 18:2 (reduced by 52%). The production of punicic acid in A. thaliana seeds was accompanied by up to a 43% increase in oleic acid content, indicating that over-expression of PgFADX led to the inhibition of the native FAD2 activity.

To enhance the accumulation of punicic acid and reduce the effect of native FAD2 inhibition observed in seeds over-expressing PgFADX, a second construct carrying PgFADX and PgFAD2 (NCJD) was developed. The fatty acid compositions of forty-five independent transgenic lines carrying NCJD construct in A. thaliana fad3/fae1 mutant were determined. The results from the best 17 A. thaliana T₂ seeds are shown in FIG. 4. Over-expression of PgFADX and PgFAD2 in A. thaliana seeds resulted in higher proportion of punicic acid compared to the seeds over-expressing only PgFADX (FIG. 3). In the best transgenic line, NCJD-33, punicic acid content was increased up to 15.24% at the expense of its precursor 18:2 (reduced by 27%). Oleic acid content in NCJD-33 seeds was reduced by 8.5% compared to non-transformed A. thaliana fad3/fae1 mutants. These results indicate that over-expression of PgFAD2 reduced significantly the inhibition effect of native FAD2 desaturase activity observed in the seeds over-expressing only PgFADX.

Since the preliminary analysis of fatty acid composition was performed on T₂ segregating seeds for the presence of the transgene(s), it was proposed that T₃ homozygous seeds of A. thaliana might contain higher proportions of punicic acid. Seeds from the T₂ lines with the highest proportion of punicic acid were thus sown and grown to obtain the T₃ seed generation.

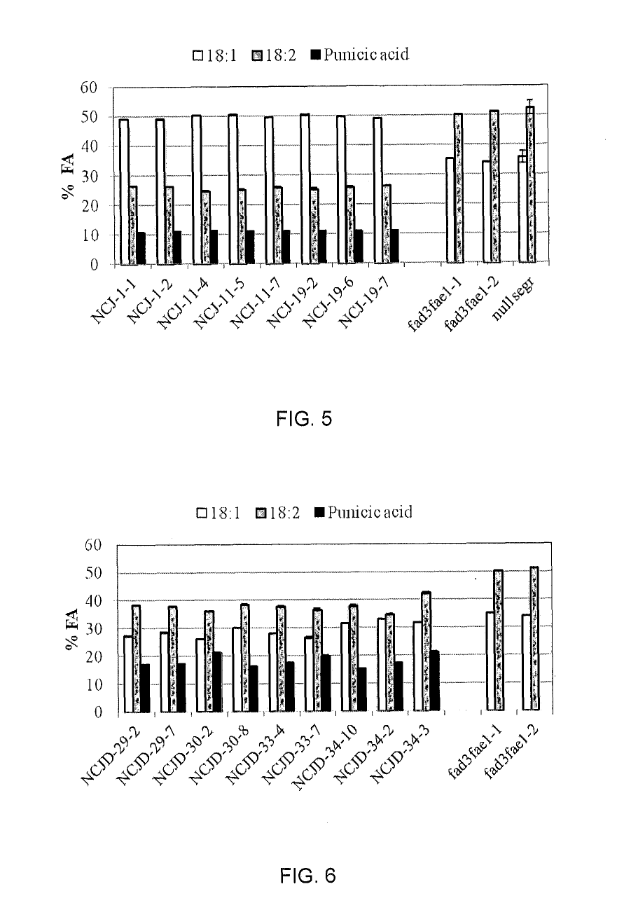

Seeds from eight T₃ homozygous transgenic lines carrying NCJ construct in high linoleic acid (52%) A. thaliana fad3/fae1 mutant background were analyzed (FIG. 5). Significant changes in fatty acid composition in comparison to the control lines (fad3/fae1) were found. The proportion of punicic acid (18:3Δ^{9Z,11E,13Z}) increased from 0% in control lines (fad3/fae1) to as high as 11.4% in the best transgenic line, NCJ-11-4. The increased proportion of punicic acid was correlated with concomitant reduction in the proportion of its precursor 18:2 (reduced by 51.3%). The production of punicic acid in A. thaliana seeds was accompanied by up to a 45% increase in oleic acid content, indicating that over-expression of PgFADX led to the inhibition of the native FAD2 activity.

To enhance the accumulation of punicic acid and reduce the effect of native FAD2 inhibition observed in seeds over-expressing PgFADX, a second construct carrying PgFADX and PgFAD2 (NCJD) was developed. The fatty acid compositions of nine T₃ transgenic lines carrying NCJD construct in A. thaliana fad3/fae1 mutant are shown in FIG. 6. Over-expression of PgFADX and PgFAD2 in A. thaliana seeds resulted in higher proportion of punicic acid compared to the seeds over-expressing only PgFADX (FIG. 5). In the best transgenic lines, NCJD-33-2 and 34-3, punicic acid content was increased up to 21% at the expense of its precursor 18:2 (reduced by 28.6%). Oleic acid content in the best transgenic NCJD-33-2 seed line was reduced by 24% compared to non-transformed A. thaliana fad3/fae1 mutant, indicating that over-expression of PgFAD2 reduced significantly the inhibition effect of native FAD2 activity observed in the seeds over-expressing only PgFADX.

### Example 4

### Fatty Acid Composition of the Selected Lipid Classes in P. granatum Seeds and A. thaliana Engineered to Synthesize Punicic Acid in the Seed Oil

Examination of the fatty acid content of specific lipid classes was performed for the A. thaliana line over-expressing PgFADX+PgFAD2 (NCJD-30-2) with the highest content of punicic acid (21.2%) in the T3 seeds (FIG. 9). Total lipids from seeds were extracted and separated as described earlier (Mietkiewska et al., 2011). In transgenic A. thaliana seeds NCJD-30-2, the punicic acid content of phosphatidylcholine (PC) was 12.5% which was higher than that observed in TAG (6.6%). A different situation was found in P. granatum seeds where the punicic acid content was 60% of the fatty acids in TAG, and only 0.8% of fatty acids in PC. The data indicate that in A. thaliana, an efficient mechanism of trafficking punicic acid from PC to TAG is missing.

### Example 5

### Isolation of Strategic Genes Involved in Punicic Acid Trafficking

Using a degenerate primer RT-PCR approach performed on cDNA amplified from P. granatum seeds, the following genes were isolated:

a) P. granatum Diacylglycerol Acyltransferase Type 1 (PgDGAT1):

Degenerate primers designed in the conserved region included the forward primer (YQDWWNA, SEQ ID NO: 31) and reverse primer (HELCIAVP, SEQ ID NO: 32) which were used to amplify a 190 bp PCR internal fragment of a putative DGAT1 from P. granatum showing up to 89% of identity to plant DGAT1 amino acid sequences. The sequence of 190 bp PCR product was used to design a gene specific primer to amplify the 5′ and 3′ ends of cDNA using a SMART RACE cDNA Amplification kit (CLONTECH, Palo Alto, Calif., USA). Using the sequence information coming from the assembly of the partial sequences, the full length ORF (1623 bp) of a putative DGAT1 was amplified by PCR using the forward F8 primer (5′-ATGGCGACCTCCGACGGC-3′; SEQ ID NO: 33) and the reverse R8 primer (5′-TTACGGCCGGGAGCCTTTT-3′; SEQ ID NO: 34). The PgDGAT1 cDNA (SEQ ID NO: 1; FIG. 10A) encodes a polypeptide of 540 amino acids (SEQ ID NO: 2; FIG. 10B) that is most closely related to DGAT1 from Glycine max and Vernica fordii (70% identity to both of them). The PgDGAT1 sequence was submitted to GenBank and accorded NCBI accession #JQ478414.

b) P. granatum Diacylglycerol Acyltransferase Type 2 (PgDGAT2):

Degenerate primers designed in the conserved regions included the forward primer (VPGGVQE, SEQ ID NO: 35) and reverse primer (PMHVVVG, SEQ ID NO: 36) which were used to amplify a 276 bp PCR internal fragment of a putative DGAT2 from P. granatum showing up to 78% of identity to plant DGAT2 amino acid sequences. A similar approach as above was used to isolate full-length ORF (1005 bp) of DGAT2 from pomegranate seeds by PCR using the forward F9 primer (5′-ATGGGAGAGGAGGCGAGC-3′, SEQ ID NO: 37) and reverse R9 primer (5′-TCAGAGGATCTTCAGTTCC-3′, SEQ ID NO: 38). The P. granatum DGAT2 cDNA (SEQ ID NO: 3) encodes a polypeptide of 334 amino acids (SEQ ID NO: 4) with the highest sequence identity (71%) to DGAT2 from Olea europaea. The sequence of the PgDGAT2 homolog was submitted to GenBank and accorded NCBI accession #JQ513387.

c) P. granatum Phospholipid:Diacylglycerol Acyltransferase 1 (PgPDAT1):

Degenerate primers designed in the conserved regions included the forward primer (LCWVEHM, SEQ ID NO: 39) and reverse primer (TQSGAHV, SEQ ID NO: 40) which were used to amplify a 1.4 kb PCR internal fragment of a putative PDAT from P. granatum showing up to 82% of identity to plant PDAT homologs. A similar approach as above was used to isolate full-length ORF (2052 bp) of PDAT from pomegranate seeds by PCR using the forward F10 primer (5′-ATGGCGTTTCTCTGGCGGA-3′, SEQ ID NO: 41) and the reverse R10 primer (5′-CTAGAGTGGCAAGTCAATCC-3′, SEQ ID NO: 42). The PgPDAT1 cDNA (SEQ ID NO: 5) encodes a polypeptide of 683 amino acids (SEQ ID NO: 6) with the highest sequence identity (83%) to PDAT1 from Glycine max and Vitis vinifera (82%). The sequence of the PgPDAT1 homolog was submitted to GenBank and accorded NCBI accession #JQ513388.

### Example 6

### Functional Characterization of PgDGAT1 and PgDGAT2

To establish the function of PgDGAT1 and PgDGAT2, constructs carrying two genes were developed, namely PgFADX+DGAT1 and PgFADX+DGAT2, in the yeast expression vector pESC-URA (Agilent).

PgFADX was amplified by PCR using the forward F11 primer (5′-AATAGGATCCGAAATGGGAGCTGATGGAACA-3′; BamHI site is underlined; SEQ ID NO: 43) and the reverse R11 primer (5′-TTATGGTACCTCAGAACTTGCTCTTGAAC-3′; KpnI site is underlined; SEQ ID NO: 44), and cloned in BamHI and KpnI sites of pESC-URA to yield the pEX construct.

PgDGAT1 was amplified by PCR using the forward F12 primer (5′-GCAGAGCGGCCGCGAAATGGCGACCTCCGACGGC-3′; NotI site is underlined; SEQ ID NO: 45) and the reverse R12 primer (5′-ATATTTAAATTAATTACGGCCGGGAGCCTTTT-3′; PacI site is underlined; SEQ ID NO: 46), and cloned in the NotI and PacI sites of pEX.

PgDGAT2 was amplified by PCR using the forward. F13 primer (5′-GCAGAGCGGCCGCGAAATGGGAGAGGAGGCGAG-3′; NotI site is underlined; SEQ ID NO: 47) and the reverse R13 primer (5′-ATATTTAATTAATCAGAGGATCTTCAGTTCC-3′; PacI site is underlined; SEQ ID NO: 48); and cloned in NotI and PacI sites of pEX.

The prepared constructs were transformed into yeast cells H1246 (Sandager et al., 2002). Yeast cultures were grown at 30° C. for 48 h. Expression of the recombinant genes was induced using minimal medium containing 2% (w/v) galactose and 1% (w/v) raffinose supplemented with 100 μM of linoleic acid (18:2).

In yeast cells over-expressing only PgFADX, punicic acid was found only in the polar lipids (PL) fraction where its synthesis occurs (FIG. 13). In yeast cells over-expressing PgFADX+PgDGAT1, the punicic acid content of polar lipids was 0.82% and was higher than that observed in triacylglycerol (TAG, 0.13%). Significantly higher accumulation of punicic acid in TAG (0.4%) was found in yeast cells over-expressing PgFADX+PgDGAT2.

Without being bound by theory, these results indicate that PgDGAT1 and PgDGAT2 encode enzymes involved in the efficient trafficking of punicic acid from the origin of its synthesis (PL) to the storage lipids (TAG). PgDGAT1 and PgDGAT2 appear to be suitable strategic genes to enhance the accumulation of punicic acid to higher levels than those observed in A. thaliana plants over-expressing only PgFADX+PgFAD2.

### Example 7

### Production of Punicic Acid in A. thaliana Seeds Combining PgFADX, PgFAD2 and PgDAT2

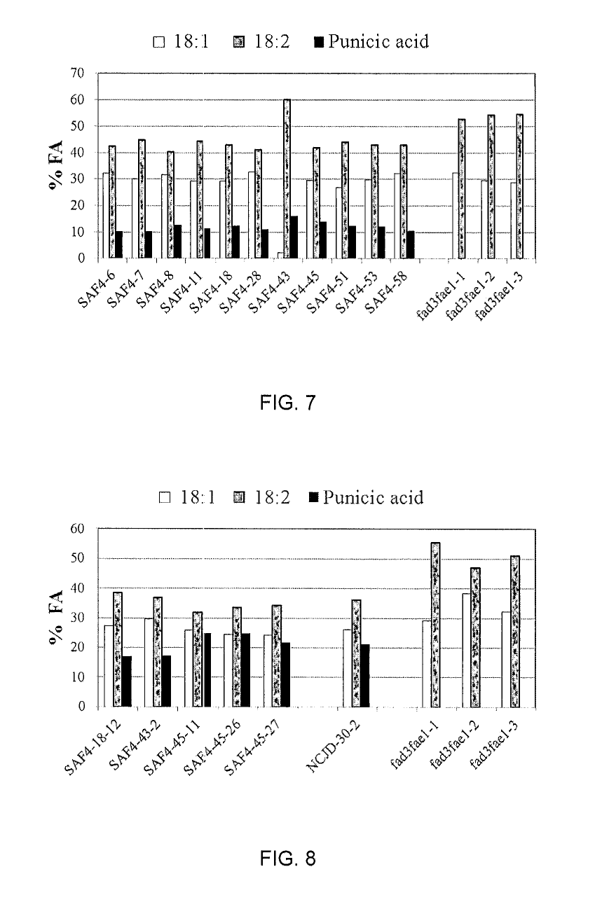

A construct carrying PgFADX, PgFAD2 and PgDGAT2 (SAF4) was developed to determine whether PgDGAT2 might increase the accumulation of punicic acid in A. thaliana over the level previously observed in lines over-expressing PgFADX and PgFAD2. Seeds from 48 independent transgenic lines carrying the SAF4 construct in high linoleic acid (52%) A. thaliana fad3/fae1 mutant background were analyzed. Results from the best 11 A. thaliana T₂ lines are shown in FIG. 7. In the transgenic lines SAF4-43 and SAF4-45 over-expressing PgFADX, PgFAD2 and PgDGAT2, punicic acid accounted for 16.02% and 13.84% respectively of total fatty acids compared to 15.24% found in the best T₂ NCJD-33 line over-expressing only PgFADX and PgFAD2.

Since the preliminary analysis of fatty acid composition was performed on T₂ segregating seeds for the presence of the transgene(s), it was proposed that T₃ seeds of A. thaliana might contain higher proportions of punicic acid. Seeds from the T₂ lines with the highest proportion of punicic acid were sown and grown in order to obtain the T₃ seed generation.

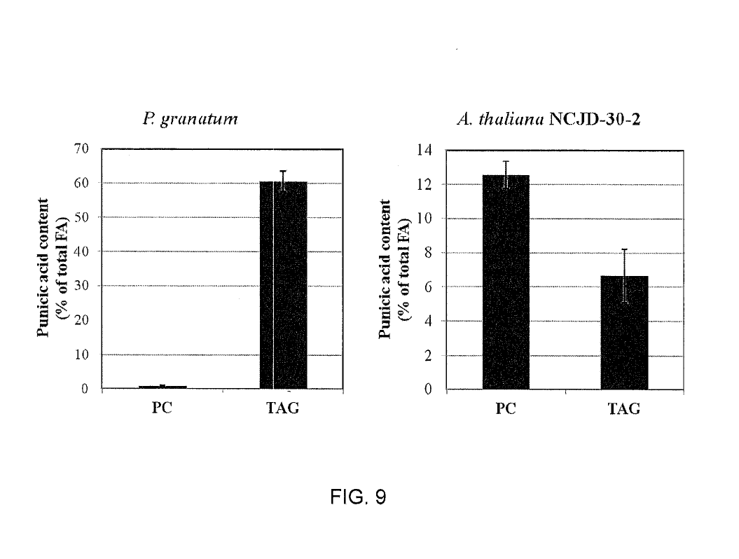

Fatty acid compositions of 5 T₃ transgenic lines carrying SAF4 construct in A. thaliana fad3/fae1 mutant are shown in FIG. 8. In T₃ transgenic lines SAF4-45-11 and SAF4-45-26, the punicic acid content increased up to 24.8% of total fatty acids in the seed oil. Over-expression of PgDGAT2 increased the punicic acid content compared to that which was present in plants over-expressing only PgFADX and PgFAD2 (NCJD-30-2; 21.2% of punicic acid). The combined over-expression of PgFADX, PgFAD2 and PgDGAT2 yielded the highest overall production of punicic acid (24.8%) in transformed A. thaliana seeds,

### REFERENCES

The following references are incorporated herein by reference (where permitted) as if reproduced in their entirety. All references are indicative of the level of skill of those skilled in the art to which this invention pertains.
Bevan, M. (1983) Binary Agrobacterium vectors for plant transformation. Nucleic Acid Res. 12, 8711-8721.
Boussetta T., Raad H., Lettéron P., Gougerot-Pocidalo M. A., Marie J. C, Driss F., and El-Benna J. (2009) Punicic acid: a conjugated linolenic acid inhibits TNFα-induced neutrophil hyperactivation and protects from experimental colon inflammation in rats. PLOS ONE 4: e6458.
Broun P. and Somerville C. (1997) Accumulation of ricinoleic, lesquerolic, and densipolic acids in seeds of transgenic Arabidopsis plants that express a fatty acyl hydroxylase cDNA from castor bean, Plant Physiol. 113: 933-942,
Burgal J., Shockey J., Lu C., Dyer J., Larson T., Graham I. and Browse J. (2008) Metabolic engineering of hydroxy fatty acid production in plants: RcDGAT2 drives dramatic increases in ricinoleate levels in seed oil. Plant Biotechnol. J. 6: 819-831.
Cahoon E. B., Dietrich C. R., Meyer K., Damude H. G., Dyer J. M, and Kinney A. J. (2006) Conjugated fatty acids accumulate to high levels in phospholipids of metabolically engineered soybean and Arabidopsis seeds. Phytochemistry 67: 1166-1176.
Cahoon E. B., Shockey J. M., Dietrich C. R., Gidda S. K, Mullen R. T, and Dyer J. M. (2007) Engineering oilseeds for sustainable production of industrial and nutritional feedstocks: solving bottlenecks in fatty acid flux. Curr. Opin. Plant Biol. 10: 236-244.
Clough S. J. and Bent A. F. (1998) Floral dip: a simplified method for Agrobacterium-mediated transformation of Arabidopsis thaliana. Plant J. 16: 735-743.
Drexler H., Spiekermann P., Meyer A., Domergue F., Zank T., Sperling P., Abbadi A. and Heinz E. (2003) Metabolic engineering of fatty acids for breeding of new oilseed crops: strategies, problems and first results. J. Plant Physiol, 160: 779-802.
Dyer J. M. and Mullen R. T. (2008) Engineering plant oils as high-value industrial feedstocks forbiorefining: the need for underpinning cell biology research. Physiol. Plant, 132:11-22.
Dyer J. M., Stymne S., Green A. G. and Carlsson A. S. (2008) High-value oils from plants. Plant J. 54: 640-655.
Goderis I. J., De Bolle M. F., François I. E., Wouters P. F., Broekaert W. F. and Cammue B. P. (2002) A set of modular plant transformation vectors allowing flexible insertion of up to six expression units. Plant Mol. Biol. 50(1):17-27.
Hornung E., Pernstich C. and Feussner I. (2002) Formation of conjugated Δ11 Δ13-double bonds by Δ12-linoleic acid (1,4)-acyl-lipid-desaturase in pomegranate seeds. Eur. J. Biochem. 269: 4852-4859.
Iwabuchi M., Kohno-Murase J. and Imamura J, (2003) Δ12-oleate desaturase-related enzymes associated with formation of conjugated trans-Δ11, cis-Δ13 double bonds, J. Biol. Chem. 278: 4603-4610.
Josefsson, L. G., Lenman, M., Ericson, M. L. and Rask, L. (1987) Structure of a gene encoding the 1.7 S storage protein, napin, from Brassica napus. J. Biol. Chem. 262, 12196-12201.
Kim N. D., Mehta R., Yu W., Neeman I., Livney T., Amichay A., Poirier D., Nicholls P., Kirby A., Jiang W., Mansel R., Ramachandran C., Rabi T., Kaplan B. and Lansky E. (2002) Chemopreventive and adjuvant therapeutic potential of pomegranate (Punica granatum) for human breast cancer. Breast Cancer Research and Treatment 71: 203-217.
Li R., Yu K., Hatanaka T. and Hildebrand D. F. (2010) Vernonia DGATs increase accumulation of epoxy fatty acids in oil. Plant Biotechnol. J. 8: 184-195.
Mietkiewska E., Brost J. M., Giblin E. M., Barton D. L. and Taylor D. C. (2007) Cloning and functional characterization of the Fatty Acid Elongase 1 (FAE1) gene from high erucic Crambe abyssinica cv. Prophet. Plant Biotechnol. J. 5: 636-645.
Mietkiewska E., Siloto R. M., Dewald J., Shah S., Brindley D. N. and Weselake R. J. (2011) Lipins from plants are phosphatidate phosphatases that restore lipid synthesis in a pah1Δ mutant strain of Saccharomyces cerevisiae. FEBS Journal 278: 764-775.
Sandager, L., Gustaysson, M. H., Stahl, U., Dahlqvist, A., Wiberg, E., Banas, A., Lenman, M., Ronne, H. and Stymne, S. (2002) Storage lipid synthesis is non-essential in yeast. J. Biol. Chem. 277: 6478-6482.
Singh S. F., Zhou X-R, Liu Q., Stymne S. and Green A. G. (2005) Metabolic engineering of new fatty acids in plants. Curr. Opin. Plant Biol. 8: 197-203.
Shockey, J. M., Gidda, S. K., Chapital, D. C., Kuan, J. C., Dhanoa, P. K., Bland J. M., Rothstein S. J., Mullen R. T. and Dyer J. M. (2006) Tung tree DGAT1 and DGAT2 have nonredundant functions in triacylglycerol biosynthesis and are localized to different subdomains of the endoplasmic reticulum. Plant Cell 18: 2294-2313.
Smith M. A., Moon H., Chowrira G. and Kunst L. (2003) Heterologous expression of a fatty acid hydroxylase gene in developing seeds of Arabidopsis thaliana. Planta 217: 507-516.
Stahl U., Banas A. and Stymne S. (1995) Plant microsomal phospholipid acyl hydrolases have selectivities for uncommon fatty acids. Plant Physiol, 107: 953-962.
Syed D., Suh Y., Afaq F. and Mukhtar H. (2008) Dietary agents for chemoprevention of prostate cancer. Cancer Lett. 265:167-176.
Thomaeus S., Carlsson A. S., Lee M. and Stymne S. (2001) Distribution of fatty acids in polar and neutral lipids during seed development in Arabidopsis thaliana genetically engineered to produce acetylenic, epoxy and hydroxy fatty acids. Plant Sci. 161: 997-1003.
van Erp H., Bates D. P., Burgal J., Shockey J. and Browse J. (2011) Castor phospholipid:diacylglycerol acyltransferase facilitates efficient metabolism of hydroxy fatty acids in transgenic Arabidopsis. Plant Physiol. 155: 683-693.
Zhou X. R., Singh S., Liu Q. and Green A. (2006) Combined transgenic expression of D12-desaturase and D12-epoxygenase in high linoleic substrate seed oil leads to increased accumulation of vernolic acid. Funct. Plant Biol. 33: 585-592.

What is claimed is:

1. An isolated polynucleotide sequence encoding a protein or polypeptide comprising or consisting of an amino acid sequence selected from SEQ ID NO: 2, 4 or 6, respective biologically active variants and biologically active portions thereof, with respective sequences having at least 85% identity thereto, and wherein the variants or portions have diacylglycerol acyltransferase type 1 (DGAT1), type 2 (DGAT2) or phospholipid diacylglycerol acyltransferase (PDAT) activity.

2. The isolated polynucleotide of claim 1, wherein the polynucleotide encodes a polypeptide having DGAT activity and comprising the amino acid sequence of SEQ ID NO: 2 or 4, or an amino acid sequence having DGAT activity and having at least 85% sequence identity therewith.

3. The isolated polynucleotide of claim 1, wherein the polynucleotide encodes a polypeptide having PDAT activity and comprising the amino acid sequence of SEQ ID NO: 6, or an amino acid sequence having PDAT activity and having at least 85% sequence identity therewith.

4. The isolated polynucleotide of claim 1, wherein the polynucleotide comprises the nucleotide sequence of SEQ ID NO: 1, 3 or 5.

5. The isolated polynucleotide of claim 1, wherein the encoded polypeptide comprises an amino acid sequence having at least 90%, at least 95%, at least 96%, at least 97%, at least 98%, or at least 99% sequence identity to one of SEQ ID NO: 2, 4 or 6.

6. The isolated polynucleotide of claim 1, wherein the polynucleotide is derived from Punica granatum.

7. A recombinant expression vector comprising a polynucleotide sequence of claim 1 operably linked with transcriptional and translational regulatory regions or sequences to provide for expression of the at least one polynucleotide sequence in a host cell.

8. A transgenic plant, plant cell, plant seed, callus, plant embryo, microspore-derived embryo, or microspore, comprising a recombinant expression vector encoding PgFADX and PgFAD2.

9. The transgenic plant, plant cell, plant seed, callus, plant embryo, microspore-derived embryo, or microspore of claim 8, which further comprises an expression vector encoding one or more of a PgDGAT and PgPDAT.

10. The transgenic plant, plant cell, plant seed, callus, plant embryo, microspore-derived embryo, or microspore of claim 9 wherein the PgDGAT or PgPDAT comprises one or more of PgDGAT1, PgDGAT2 and PgPDAT1.

11. The transgenic plant, plant cell, plant seed, callus, plant embryo, microspore-derived embryo, or microspore of claim 10, which comprises PgFADX, PgFAD2, and PgDGAT2.

12. The transgenic plant, plant cell, plant seed, callus, plant embryo, microspore-derived embryo, or microspore of claim 8, which comprises a progeny plant generated from the transgenic plant.

13. The transgenic plant, plant cell, plant seed, callus, plant embryo, microspore-derived embryo, or microspore of claim 8, which is selected from a linseed, rapeseed, canola, peanut, safflower, flax, hemp, camelina, soybean, pea, sunflower, olive, palm, oats, wheat, triticale, barley, corn, thale cress, and legume plant, plant cell, plant seed, callus, plant embryo, or microspore-derived embryo or microspore.

14. The transgenic plant, plant cell, plant seed, callus, plant embryo, microspore-derived embryo, or microspore of claim 13, which comprises Arabidopsis thaliana.

15. The transgenic plant, plant cell, plant seed, callus, plant embryo, microspore-derived embryo, or microspore of claim 14, which comprises Arabidopsis thaliana fad3/fae1 double mutant.

16. A method of increasing punicic acid production in an oilseed plant, comprising the step of transforming an oilseed plant to over-express PgFADX and PgFAD2.

17. The method of claim 16, further comprising the step of transforming the plant to over-express a gene encoding a DGAT and/or a PDAT.

18. The method of claim 17 wherein the DGAT or PDAT comprises a PgDGAT or PgPDAT.

19. The method of claim 18, wherein PgFADX, PgFAD2, and one or more of PgDGAT1, PgDGAT2 and PgPDAT1 are over-expressed in the plant.

20. The method of claim 19, wherein PgFADX, PgFAD2, and PgDGAT2 are over-expressed in the plant.

21. The method of claim 16 wherein the oilseed plant comprises linseed, rapeseed, canola, peanut, safflower, flax, hemp, camelina, soybean, pea, sunflower, olive, palm, oats, wheat, triticale, barley, corn, thale cress, or legume.

22. The method of claim 21, wherein the plant comprises Arabidopsis thaliana.

23. The method of claim 22 wherein the plant comprises an Arabidopsis thaliana fad3/fae1 double mutant.
